(19) United States
(12) Patent Application Publication
(10) Pub. No.: US 2010/0291563 A1
(43) Pub. Date: Nov. 18, 2010

(54) ASSESSING TISSUE REJECTION

(76) Inventor: Philip F. Halloran, Edmonton (CA)

Correspondence Address: FISH & RICHARDSON P.C., PO BOX 1022, MINNEAPOLIS, MN, 55440-1022, US

(21) Appl. No.: 12/666,997

(22) PCT Filed: Jun. 25, 2008
(86) PCT No.: PCT/CA2008/001180

§ 371 Date: Jun. 18, 2010

Related U.S. Application Data
(60) Provisional application No. 60/937,733, filed on Jun. 29, 2007.

(51) Int. Cl. C12Q 1/68; C12Q 1/02; G01N 33/68

(52) U.S. Cl. 435/6; 435/29; 435/7.1

(57) ABSTRACT

This document relates to methods and materials involved in assessing tissue rejection (e.g., organ rejection) in mammals. For example, methods and materials involved in detecting tissue rejection (e.g., kidney rejection) are provided, as are methods and materials for determining the extent of rejection in mammals (e.g., humans).

### BACKGROUND

1. Technical Field

This document relates to methods and materials involved in assessing tissue rejection (e.g., organ rejection) in mammals. For example, this document relates to methods and materials involved in detecting tissue rejection (e.g., kidney rejection) in mammals and determining the burden or extent of rejection in mammals (e.g., humans).

2. Background Information

The diagnosis of allograft rejection remains an important issue in kidney transplantation. Rejection can manifest as an acute episode or as subtle loss of function, proteinuria, scarring, and graft loss (Meier-Kriesche et al., Am J Transplant, 4(3):378-383 (2004)). Two mechanisms of rejection are recognized in the Banff histologic classification: T cell mediated rejection (TCMR) and antibody-mediated rejection (ABMR; Solez et al., Am J Transplant, 7(3):518-526 (2007); Racusen et al., Am J Transplant, 4(10):1562-1566 (2004)). TCMR can be diagnosed by scoring interstitial inflammation (i), tubulitis (t), and vasculitis (v) and its association with infiltration by cytotoxic T lymphocytes (CTL). A hallmark of ABMR is C4d deposition in peritubular capillaries (Racusen et al., Am J Transplant, 3(6):708-714 (2003)).

### SUMMARY

This document provides methods and materials involved in assessing tissue rejection (e.g., organ rejection) in mammals. For example, this document provides methods and materials involved in the early detection of tissue rejection (e.g., kidney rejection) and the assessment of the extent of rejection of a tissue, e.g., a transplanted organ, in a mammal. Early diagnosis of patients rejecting transplanted tissue (e.g., a kidney) can help clinicians determine appropriate treatments for those patients. For example, a clinician who diagnoses a patient as rejecting transplanted tissue can treat that patient with medication that suppresses tissue rejection (e.g., an immunosuppressant).

Despite international consensus, histologic grading of rejection is poorly reproducible (Marcussen et al., Transplant, 60:1083-1089 (1995); Furness et al., Am J Surg Pathol, 27(6):805-810 (2003); Furness et al., Nephrol Dial Transplant, 12(5):995-100 (1997); Furness et al., Histopathology, 35(5):461-467 (1999)). This is particularly problematic at the important interface that separates TCMR from borderline changes, which is the point that also defines where treatment decisions are made (Furness et al., Nephrol Dial Transplant, 12(5):995-100 (1997)). Moreover, i-lesions and t-lesions are not specific for TCMR, and are often found in stable kidney transplants where their significance is unclear (Colvin, N Eng J Med, 349(24):2288-2290 (2003); Mengel et al., Am J Transplant (2007)). Other limitations are inherent in diagnostic pathology of rejection, including sampling error, intra-observer variation, and a shortage of trained pathologists. In addition, describing morphology does not produce a picture of active events such as active inflammation and active injury, and provides a qualitative assessment of tissue after damage has occurred or even progressed. For example, although fibrosis can be observed with pathology, pre-fibrotic events are not detectable with a standard Banff pathology assessment. Better methods are needed for assessing transplant rejection.

This document is based in part on the discovery of nucleic acids that are differentially expressed in kidney biopsies with TCMR, biopsies with acute tubular necrosis (ATN), and normal kidneys. The levels of these nucleic acids and/or polypeptides encoded by these nucleic acids can be used to determine whether tissue transplanted into a mammal is being rejected and the extent of that rejection. For example, transplanted tissue containing cells expressing one third or more of the nucleic acids listed in Table 2 at a level that is higher than the average level observed in normal kidney cells can be classified as being rejected. In some cases, transplanted tissue containing cells expressing one third or more of the polypeptides encoded by nucleic acids listed in Table 2 at a level that is higher than the average level observed in normal kidney cells can be classified as being rejected. The levels of multiple nucleic acids or polypeptides can be detected simultaneously using nucleic acid or polypeptide arrays.

In general, this document features a method for detecting tissue rejection. The method comprises, or consists essentially of, determining whether or not tissue transplanted into a human contains cells having a human transplant rejection profile, where the presence of the cells indicates the presence of rejection. The tissue can be kidney tissue. The tissue can be a kidney. The method can comprise using kidney cells obtained from a biopsy to assess the presence or absence of the human transplant rejection profile. The determining step can comprise analyzing nucleic acids. The determining step can comprise analyzing polypeptides.

In another aspect, this document features a method for assessing tissue rejection. The method comprises, or consists essentially of, determining the mean expression of quantitative CD8 CATs in cells from tissue transplanted into a human, where a greater difference between the mean expression of quantitative CD8 CATs and the mean of corresponding reference levels indicates a greater extent of rejection. The tissue can be kidney tissue. The tissue can be a kidney. The method can comprise using kidney cells obtained from a biopsy to determine the mean expression of quantitative CD8 CATs. The determining step can comprise analyzing nucleic acids. The determining step can comprise analyzing polypeptides.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic representation of the algorithm used to identify CD8 cytotoxic T lymphocyte-associated transcripts (CATs).

FIG. 2 is a graph plotting expression of CD8 CATs in human kidney biopsies from nephrectomy and TCMR cases. Each line graph depicts one of eight CD8 CAT subsets. Four subsets (CD8 CAT 1-10, CD8 CAT 11-20, CD8 CAT 21-30, and CD8 CAT 31-40) represent the top 40 CD8 CATs with the highest expression values in CD8 CTL, and four other subsets (CD8 CAT 167-176, CD8 CAT 177-186, CD8 CAT 187-196, and CD8 CAT 197-206) represent the bottom 40 CD8 CATs with the lowest expression values in CD8 CTL. Values displayed are the fold increase in signal compared to the mean nephrectomy score.

FIG. 3A is a graph plotting the geometric mean quantitative CAT signal versus the CD8⁺CTL RNA dilution ratio. FIG. 3B is a graph plotting the predicted CD8⁺CTL dilution ratio in kidney biopsies from untreated TCMR cases, treated TCMR cases, and ATN cases, normalized to normal nephrectomy samples. FIG. 3C is a graph plotting the CD8⁺CTL RNA ratio for nephrectomy, TCMR, treated TCMR, and ATN samples.

### DETAILED DESCRIPTION

This document provides methods and materials related to assessing tissue rejection (e.g., organ rejection). For example, this document provides methods and materials that can be used to identify a mammal (e.g., a human) as having transplanted tissue that is being rejected. A human can be identified as having transplanted tissue that is being rejected if it is determined that the transplanted tissue in the human contains cells having a human transplant rejection profile, a human CD8 cytotoxic T lymphocyte-associated profile, or a quantitative human CD8 cytotoxic T lymphocyte-associated profile. In some cases, a human can be identified as having transplanted tissue that is being rejected if it is determined that the transplanted tissue in the human contains cells having a mean human transplant rejection profile, a mean human CD8 cytotoxic T lymphocyte-associated profile, or a mean quantitative human CD8 cytotoxic T lymphocyte-associated profile. For the purposes of this document, the term “human transplant rejection profile” as used herein refers to a nucleic acid or polypeptide profile in a sample (e.g., a sample of transplanted tissue) where one or more than one (e.g., 1, 2, 3, 4, 5, 6, 7, 8, 9, 10, 11, 12, 13, 14, 15, 16, 17, 18, 19, 20, 21, 22, 23, 24, or more) of the nucleic acids or polypeptides encoded by the nucleic acids listed in Table 2, or listed in the first forty rows of Table 1, is present at an elevated level. For the purposes of this document, the term “human CD8 cytotoxic T lymphocyte-associated profile” as used herein refers to a nucleic acid or polypeptide profile in a sample where one third or more of the nucleic acids or polypeptides encoded by the nucleic acids listed in the first forty rows of Table 1 are present at an elevated level. For example, a human CD8 cytotoxic T lymphocyte-associated profile can be a nucleic acid or polypeptide profile in a sample where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in the first forty rows of Table 1 are present at an elevated level. For the purposes of this document, the term “quantitative human CD8 cytotoxic T lymphocyte-associated profile” as used herein refers to a nucleic acid or polypeptide profile in a sample where one third or more of the nucleic acids or polypeptides encoded by the nucleic acids listed in Table 2 are present at an elevated level. For example, a quantitative human CD8 cytotoxic T lymphocyte-associated profile can be a nucleic acid or polypeptide profile in a sample where 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 2 are present at an elevated level.

For the purposes of this document, the term “mean human transplant rejection profile” as used herein refers to a nucleic acid or polypeptide profile in a sample where the mean expression level of more than one (e.g., 2, 3, 4, 5, 6, 7, 8, 9, 10, 11, 12, 13, 14, 15, 16, 17, 18, 19, 20, 21, 22, 23, 24, 25, 26, 27, or more) of the nucleic acids or polypeptides encoded by the nucleic acids listed in Table 2, or listed in the first forty rows of Table 1, is elevated. For purposes of this document, the term “mean human CD8 cytotoxic T lymphocyte-associated profile” refers to a nucleic acid or polypeptide profile in a sample where the mean expression level of one third or more of the nucleic acids or polypeptides encoded by the nucleic acids listed in the first forty rows of Table 1 is elevated. For example, a mean human CD8 cytotoxic T lymphocyte-associated profile can be a nucleic acid or polypeptide profile in a sample where the mean expression level of 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in the first forty rows of Table 1 is elevated. For the purposes of this document, the term “mean quantitative human CD8 cytotoxic T lymphocyte-associated profile” as used herein refers to a nucleic acid or polypeptide profile in a sample where the mean expression level of one third or more of the nucleic acids or polypeptides encoded by the nucleic acids listed in Table 2 is elevated. For example, a mean quantitative human CD8 cytotoxic T lymphocyte-associated profile can be a nucleic acid or polypeptide profile in a sample where the mean expression level of 34%, 35%, 36%, 37%, 38%, 39%, 40%, 45%, 50%, 55%, 60%, 65%, 70%, 75%, 80%, 85%, 90%, 95%, or 100% of the nucleic acids or polypeptides encoded by the nucleic acids listed in Table 2 is elevated.

The methods and materials provided herein can be used to detect tissue rejection in any mammal such as a human, monkey, horse, dog, cat, cow, pig, mouse, or rat. In addition, the methods and materials provided herein can be used to detect rejection of any type of transplanted tissue including, without limitation, kidney, heart, liver, pancreas, and lung tissue. For example, the methods and materials provided herein can be used to determine whether or not a human who received a kidney transplant is rejecting that transplanted kidney and to what degree that rejection is occurring.

Any type of sample containing cells can be used to determine whether or not transplanted tissue is being rejected in a mammal. For example, biopsy (e.g., punch biopsy, aspiration biopsy, excision biopsy, needle biopsy, or shave biopsy), tissue section, lymph fluid, and blood samples can be used. In some cases, a tissue biopsy sample can be obtained directly from the transplanted tissue. In some cases, a lymph fluid sample can be obtained from one or more lymph vessels that drain from the transplanted tissue.

The term “elevated level” as used herein with respect to the level of a nucleic acid or polypeptide encoded by a nucleic acid listed in Table 1 or Table 2 is any level that is greater than a reference level for that nucleic acid or polypeptide. The term “reference level” as used herein with respect to a nucleic acid or polypeptide encoded by a nucleic acid listed in Table 1 or Table 2 is the level of that nucleic acid or polypeptide typically expressed by cells in tissues that are free of rejection. For example, a reference level of a nucleic acid or polypeptide can be the average expression level of that nucleic acid or polypeptide, respectively, in cells isolated from kidney tissue that has not been transplanted into a mammal. Any number of samples can be used to determine a reference level. For example, cells obtained from one or more healthy mammals (e.g., at least 5, 10, 15, 25, 50, 75, 100, or more healthy mammals) can be used to determine a reference level. It will be appreciated that levels from comparable samples are used when determining whether or not a particular level is an elevated level. For example, levels from one type of cells are compared to reference levels from the same type of cells. In addition, levels measured by comparable techniques are used when determining whether or not a particular level is an elevated level.

An elevated level of a nucleic acid or polypeptide encoded by a nucleic acid listed in Table 1 (e.g., in the first forty rows of Table 1) or in Table 2 can be any level provided that the level is greater than a corresponding reference level for that nucleic acid or polypeptide. For example, an elevated level of a nucleic acid or polypeptide encoded by a nucleic acid listed in Table 1 or Table 2 can be 1.1, 1.2, 1.3, 1.4, 1.5, 1.6, 1.7, 1.8, 1.9, 2.0, 2.2, 2.4, 2.6, 2.8, 3, 3.3, 3.6, 4, 4.5, 5, 5.5, 6, 7, 8, 9, 10, 15, 20, or more times greater than the reference level for that nucleic acid or polypeptide, respectively. In addition, a reference level can be any amount. For example, a reference level can be zero. In this case, any level greater than zero would be an elevated level.

Any appropriate method can be used to determine the level of a nucleic acid or polypeptide encoded by a nucleic acid listed in Table 1 or Table 2 in a sample. For example, quantitative PCR, in situ hybridization, or microarray technology can be used to measure the level of a nucleic acid listed in Table 1 or Table 2. In some cases, polypeptide detection methods, such as immunochemistry techniques, can be used to measure the level of a polypeptide encoded by a nucleic acid listed in Table 1 or Table 2. For example, antibodies specific for a polypeptide encoded by a nucleic acid listed in Table 1 or Table 2 can be used to determine the level of the polypeptide in a sample.

Once the level of a nucleic acid or polypeptide encoded by a nucleic acid listed in Table 1 or Table 2 is determined in a sample from a mammal, then the level can be compared to a reference level for that nucleic acid or polypeptide and used to assess tissue rejection in the mammal. A level of one or more than one nucleic acid or polypeptide encoded by a nucleic acid listed in Table 2, or in the first forty rows of Table 1, that is higher in a sample from a mammal than the corresponding one or more than one reference level can indicate that the mammal comprises transplanted tissue that is being rejected. For example, the presence of one third or more of the nucleic acids or polypeptides encoded by the nucleic acids listed in Table 2 at levels higher than the corresponding reference levels in a sample from a mammal can indicate that the mammal comprises transplanted tissue that is being rejected. In some cases, a level of one or more than one nucleic acid or polypeptide encoded by a nucleic acid listed in Table 2, or in the first forty rows of Table 1, that is higher in a sample from a mammal than the corresponding one or more than one reference level can indicate that the mammal is susceptible to tissue rejection.

In some cases, the mean (e.g., geometric mean) of the expression levels of more than one nucleic acid or polypeptide encoded by a nucleic acid listed in Table 2 in a sample from a mammal can be used to detect tissue rejection in a mammal. For example, the mean of the expression levels of one third or more (e.g., 35%, 45%, 55%, 65%, 75%, 85%, 95%, or 100%) of the nucleic acids or polypeptides encoded by the nucleic acids listed in Table 2 can be used to detect tissue rejection in a mammal. Such a mean expression level in a sample from a mammal (e.g., a mammal having transplanted tissue) can be compared to the mean of corresponding reference levels to determine whether or not the mean expression level in the sample from the mammal is elevated. An elevated mean expression level can indicate that the mammal has transplanted tissue that is being rejected. In some cases, the mean (e.g., geometric mean) of the expression levels of more than one nucleic acid or polypeptide encoded by a nucleic acid listed in the first forty rows of Table 1 in a sample from a mammal can be used to detect tissue rejection in the mammal. For example, the mean of the expression level of one third or more (e.g., 35%, 45%, 55%, 65%, 75%, 85%, 95%, or 100%) of the nucleic acids or polypeptides encoded by nucleic acids listed in the first forty rows of Table 1 in a sample from a mammal can be used to detect tissue rejection.

In some cases, the value of the mean of the expression levels of more than one nucleic acid listed in Table 2 (e.g., at least one third of the nucleic acids listed in Table 2, or all of the nucleic acids listed in Table 2) can be inserted into an equation defining a standard curve to estimate the cytotoxic T lymphocyte burden in a sample from a mammal. A standard curve can be generated by analyzing a series of dilutions of an RNA sample obtained from CD8 CTL cells from one or more healthy donors. The RNA sample can be diluted into increasing amounts of RNA isolated from a nephrectomy sample from a mammal free of tissue rejection. Each sample in the dilution series can be analyzed to determine the expression levels of more than one nucleic acid listed in Table 2 (e.g., at least one third of the nucleic acids listed in Table 2, or all of the nucleic acids listed in Table 2), and the mean expression level can be plotted against the dilution factor of the RNA sample. The mean expression level of the same nucleic acids used to generate a standard curve in a sample from a mammal can then be inserted into the equation defining the standard curve, and the equation can be solved for the dilution of the CD8 CTL RNA sample to estimate the CTL burden in the sample from the mammal. An estimated CTL burden in a sample from a mammal that is higher than a corresponding reference value can indicate that transplanted tissue in the mammal is being rejected, or is susceptible to being rejected. A reference value can be, for example, an average of estimated CTL burden values in more than one corresponding control sample obtained from more than one mammal that does not have transplanted tissue.

In some cases, the mean (e.g., geometric mean) of the expression levels of more than one nucleic acid or polypeptide encoded by a nucleic acid listed in Table 2 in a sample from a mammal can be used to assess the extent of rejection of a tissue in the mammal. For example, the mean of the expression levels of one third or more (e.g., 35%, 45%, 55%, 65%, 75%, 85%, 95%, or 100%) of the nucleic acids or polypeptides encoded by the nucleic acids listed in Table 2 in a sample from a mammal can be used to assess the extent of rejection of a tissue in the mammal. Such a mean expression level in a sample from a mammal (e.g., a mammal having transplanted tissue) can be compared to the mean of corresponding reference levels. For example, a mean expression level of GZMA and CD2 can be compared to the mean of reference levels of GZMA and CD2. The greater the difference between the mean of the expression levels of more than one nucleic acid or polypeptide encoded by a nucleic acid listed in Table 2 and the mean of corresponding reference levels, the greater the extent of the rejection. For the purposes of this document, the mean of the expression levels of one third or more (e.g., 35%, 45%, 55%, 65%, 75%, 85%, 95%, or 100%) of the nucleic acids or polypeptides encoded by the nucleic acids listed in Table 2 is referred to herein as “mean expression of quantitative CD8 CATs.”

In some cases, the mean (e.g., geometric mean) of the expression levels of more than one nucleic acid or polypeptide encoded by a nucleic acid listed in the first forty rows of Table 1 in a sample from a mammal can be used to assess the extent of rejection of a tissue in the mammal. For example, the mean of the expression level of one third or more (e.g., 35%, 45%, 55%, 65%, 75%, 85%, 95%, or 100%) of the nucleic acids or polypeptides encoded by nucleic acids listed in the first forty rows of Table 1 in a sample from a mammal can be used to assess the extent of rejection of a tissue in the mammal. Such a mean expression level in a sample from a mammal (e.g., a mammal having transplanted tissue) can be compared to the mean of corresponding reference levels. The greater the difference between the mean of the expression levels of more than one nucleic acid or polypeptide encoded by a nucleic acid listed in the first forty rows of Table 1 and the mean of corresponding reference levels, the greater the extent of the rejection. For the purposes of this document, the mean of the expression levels of one third or more (e.g., 35%, 45%, 55%, 65%, 75%, 85%, 95%, or 100%) of the nucleic acids or polypeptides encoded by the nucleic acids listed in the first forty rows of Table 1 is referred to herein as “mean expression of CD8 CATs.”

In some cases, the expression level of one nucleic acid or polypeptide encoded by a nucleic acid listed in Table 2 in a sample from a mammal can be used to assess the extent of rejection of a tissue in the mammal. The expression level of the nucleic acid or polypeptide encoded by a nucleic acid listed in Table 2 can be compared to the corresponding reference level. The greater the difference between the expression level of the nucleic acid or polypeptide encoded by a nucleic acid listed in Table 2 and the corresponding reference level, the greater the extent of the rejection.

The methods and materials provided herein can be used at any time following a tissue transplantation to determine whether or not the transplanted tissue will be rejected. For example, a sample obtained from transplanted tissue at any time following the tissue transplantation can be assessed for the presence of cells expressing an elevated level of one or more nucleic acids or polypeptides encoded by nucleic acids provided herein. In some cases, a sample can be obtained from transplanted tissue 1, 2, 3, 4, 5, 6, 7, 8, 9, 10, 11, 12, or more hours after the transplanted tissue was transplanted. In some cases, a sample can be obtained from transplanted tissue one or more days (e.g., 2, 3, 4, 5, 6, 7, 8, 9, 10, 15, 20, 25, 30, 35, 40, 45, 50, 55, 60, or more days) after the transplanted tissue was transplanted. For example, a sample can be obtained from transplanted tissue 2 to 7 days (e.g., 4 to 6 days) after transplantation and assessed for the presence of cells expressing an elevated level of a nucleic acid or polypeptide encoded by a nucleic acid provided herein. Typically, a biopsy can be obtained any time after transplantation if a patient experiences reduced graft function.

This document also provides methods and materials to assist medical or research professionals in determining whether or not a mammal is undergoing tissue rejection. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of one or more nucleic acids or polypeptides encoded by nucleic acids listed in Table 1 or Table 2 in a sample, and (2) communicating information about that level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document also provides nucleic acid arrays. The arrays provided herein can be two-dimensional arrays, and can contain at least two different nucleic acid molecules (e.g., at least three, at least five, at least ten, at least 20, at least 30, at least 40, at least 50, or at least 60 different nucleic acid molecules). Each nucleic acid molecule can have any length. For example, each nucleic acid molecule can be between 10 and 250 nucleotides (e.g., between 12 and 200, 14 and 175, 15 and 150, 16 and 125, 18 and 100, 20 and 75, or 25 and 50 nucleotides) in length. In some cases, an array can contain one or more cDNA molecules encoding, for example, partial or entire polypeptides. In addition, each nucleic acid molecule can have any sequence. For example, the nucleic acid molecules of the arrays provided herein can contain sequences that are present within nucleic acids listed in Table 1 or Table 2.

In some cases, at least 25% (e.g., at least 30%, at least 40%, at least 50%, at least 60%, at least 75%, at least 80%, at least 90%, at least 95%, or 100%) of the nucleic acid molecules of an array provided herein contain a sequence that is (1) at least 10 nucleotides (e.g., at least 11, 12, 13, 14, 15, 16, 17, 18, 19, 20, 25, or more nucleotides) in length and (2) at least about 95 percent (e.g., at least about 96, 97, 98, 99, or 100) percent identical, over that length, to a sequence present within a nucleic acid listed in Table 1 (e.g., the first forty rows of Table 1) or in Table 2. For example, an array can contain 60 nucleic acid molecules located in known positions, where each of the 60 nucleic acid molecules is 100 nucleotides in length while containing a sequence that is (1) 90 nucleotides is length, and (2) 100 percent identical, over that 90 nucleotide length, to a sequence of a nucleic acid listed in Table 1 or Table 2. A nucleic acid molecule of an array provided herein can contain a sequence present within a nucleic acid listed in Table 1 or Table 2 where that sequence contains one or more (e.g., one, two, three, four, or more) mismatches.

The nucleic acid arrays provided herein can contain nucleic acid molecules attached to any suitable surface (e.g., plastic, nylon, or glass). In addition, any appropriate method can be used to make a nucleic acid array. For example, spotting techniques and in situ synthesis techniques can be used to make nucleic acid arrays. Further, the methods disclosed in U.S. Pat. Nos. 5,744,305 and 5,143,854 can be used to make nucleic acid arrays.

This document also provides arrays for detecting polypeptides. The arrays provided herein can be two-dimensional arrays, and can contain at least two different polypeptides capable of detecting polypeptides, such as antibodies (e.g., at least three, at least five, at least ten, at least 20, at least 30, at least 40, at least 50, or at least 60 different polypeptides capable of detecting polypeptides). The arrays provided herein also can contain multiple copies of each of many different polypeptides. In addition, the arrays for detecting polypeptides provided herein can contain polypeptides attached to any suitable surface (e.g., plastic, nylon, or glass).

A polypeptide capable of detecting a polypeptide can be naturally occurring, recombinant, or synthetic. The polypeptides immobilized on an array also can be antibodies. An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type, (e.g., IgG, IgM, IgD, IgA or IgY), class (e.g., IgG1, IgG4, or IgA2), or subclass. In addition, an antibody can be from any animal including birds and mammals. For example, an antibody can be a mouse, chicken, human, rabbit, sheep, or goat antibody. Such an antibody can be capable of binding specifically to a polypeptide encoded by a nucleic acid listed in Table 1 or Table 2. The polypeptides immobilized on the array can be members of a family such as a receptor family.

Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a nucleic acid encoding the partial antibody sequence. In some cases, an antibody fragment can be enzymatically or chemically produced by fragmentation of an intact antibody. In addition, numerous antibodies are available commercially. An antibody directed against a polypeptide encoded by a nucleic acid listed in Table 1 or Table 2 can bind the polypeptide at an affinity of at least 10⁴ mol⁻¹ (e.g., at least 10⁵, 10⁶, 10⁷, 10⁸, 10⁹, 10¹⁰, 10¹¹, or 10¹² mol⁻¹).

Any method can be used to make an array for detecting polypeptides. For example, methods disclosed in U.S. Pat. No. 6,630,358 can be used to make arrays for detecting polypeptides. Arrays for detecting polypeptides can also be obtained commercially, such as from Panomics, Redwood City, Calif.

The invention will be further described in the following examples, which do not limit the scope of the invention described in the claims.

### EXAMPLES

### Example 1

### Characterizing Human Cytotoxic T Lymphocyte-Associated Transcripts (CATs)

Cell cultures: Cell cultures were maintained in complete RPMI (RPMI 1640 supplemented with 10% FBS (Invitrogen Life Technologies, Carlsbad, Calif.), 2 mM L-glutamine, β-mercaptoethanol, non-essential amino acids, sodium pyruvate, and antibiotic-antimycotic solution). The cultures were incubated at 37° C. in the presence of 5% CO₂.

Isolation and generation of cell populations: Whole blood samples were obtained from healthy volunteers. Peripheral blood mononuclear cells (PBMCs) were isolated from the whole blood samples by density gradient centrifugation using Ficoll® (GE Healthcare, Piscataway, N.J.). The PBMCs were used to prepare purified cell populations. Effector CD4 and CD8 cells were generated through several rounds of MLR stimulations. PBMCs were first cultured at a ratio of 1:1 with RPMI8866 cells treated with mitomycin (Sigma, St. Louis, Mo.). The mitomycin-treated RPMI8866 cells served as stimulators. During subsequent rounds of MLR stimulations, PBMCs were cultured at a ratio of 1:3 with mitomycin-treated RPMI8866 cells. Recombinant human IL-2 (eBioscience, San Diego, Calif.) was added to MLR cultures at a concentration of 50 U/mL. After four rounds of MLR, live cells were collected by density gradient centrifugation using Ficoll®, followed by CD4 and CD8 cell purification using EasySep® negative selection kits (StemCell, Vancouver, B.C., Canada), according to the manufacturer's instructions. The cell purity ranged from 92-98%, as assessed by flow cytometry. Upon re-stimulation, 95±3% of CD8⁺ CTLs stained positive for intracellular GzmB. In addition, 96±2% of CD4⁺ and 90±3% of CD8⁺CTLs stained positive for IFN-γ. These results confirmed that the cells had an effector phenotype.

B cells and NK cells were purified from PBMCs using EasySep® negative selection kits (StemCell). Greater than 97% of B cells were CD19⁺, and 90-98% of NK cells were CD56⁺CD3⁻. Human NK cells were selected from donors with similar ratios of CD56⁺/CD56^{bright} NK cells, suggestive of a cytolytic NK phenotype (Nagler et al., J Immunol., 143:3183-3191 (1989)).

Monocytes were isolated using the EasySep® Human CD14 Positive Selection Kit (StemCell). The monocytes were resuspended in complete RPMI and allowed to adhere to 100 mm plates (BD Falcon). The cells were left untreated or were treated with 500 U/mL of recombinant human IFN-γ (eBioscience) for 24 hours.

Microarrays and RNA preparation: Following homogenization of cells in 0.5 mL of Trizol reagent (Invitrogen), total RNA was extracted and purified using the RNeasy Micro Kit (Qiagen, Mississauga, Ontario, Canada). RNA (1-2 μg) was labeled using a GeneChip® HT One-Cycle Target Labeling and Controls Kit (Affymetrix, Santa Clara, Calif.), according to the manufacturer's protocol. The quality of labeled cRNA was assessed using an Agilent 2100 Bioanalyzer (Agilent, Palo Alto, Calif.), and the RNA integrity number was greater than seven. Labeled cRNA was hybridized to a Human Genome U133 Plus 2.0 Array (Affymetrix), according to the manufacturer's instructions. Arrays were scanned using a GeneArray Scanner (Affymetrix) and processed with GeneChip Operating Software Version 1.4.0 (Affymetrix).

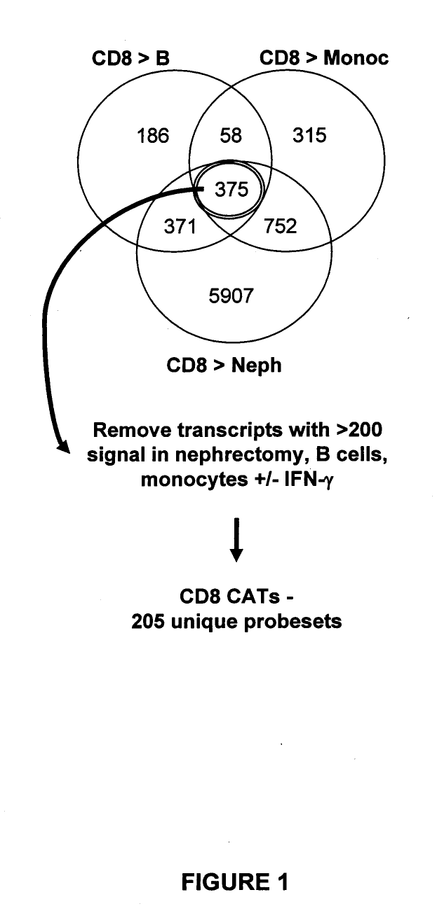

Microarray data pre-processing and selection of transcript sets: Data files were preprocessed using robust multi-chip averaging in Bioconductor version 1.9, R version 2.4, and subjected to variance-based filtering (Gentleman et al., Bioinformatics and Computational Biology Solutions Using R and Bioconductor, XII ed. (Springer 2005)), as described elsewhere (Famulski et al., Am J Transplant., 6:1342-1354 (2006)). For selection of over-expressed transcripts for each cell type from filtered data, transcript expression values had to be significant at a false discovery rate (FDR) of 0.01 in cells relative to nephrectomy samples (Smyth, Statistical Application in Genetics and Molecular Biology, 21204; 3:Article 3; Famulski et al., Am J Transplant., 6:1342-1354 (2006)). A description of the algorithms is presented in FIG. 1. Pre-processed data were imported into GeneSpring™ GX 7.3 software (Agilent, Palo Alto, Calif.) for further analyses. Gene expression was analyzed as fold increase over controls. Averaged fold changes were calculated as the geometric means, unless stated otherwise.

Real-time RT-PCR: Expression of selected transcripts (CD8A, IFNG, PRF1, GZMK, and GZMB) was confirmed by real-time RT-PCR using TaqMan Gene Expression Assays (Applied Biosystems, Foster City, Calif.). The calculated efficiencies of the gene expression assays were greater than 90%. The difference between the efficiency of the assay for each gene of interest and the efficiency of the assay for an endogenous control gene was less than 3%. The Pearson correlation of quantitative CAT set mean microarray signal with the RT-PCR results for the selected transcripts was >0.744 (p<0.001; see Example 2).

### Results

A list of mouse cytotoxic T lymphocyte-associated transcripts (CATs) with increased expression during the course of kidney T cell-mediated rejection (TCMR) was established (Einecke et al., Am J Transplant., 5:1827-1836 (2005)). Mouse CATs were derived from cultured CD8 cytotoxic T lymphocytes (CTL), and their expression was not altered during rejection in B cell-deficient hosts. The activation state of T cells infiltrating mouse CBA kidney allografts undergoing rejection in C57BL/6 hosts seven days post-transplant was examined in the absence of immunosuppression. CD4 and CD8 T cells from transplants and host spleens were stained for CD44 and CD62L and analyzed by flow cytometry. This analysis showed that strong CD4 and CD8 T cell responses were generated in host spleens and that only activated/effector T cells (CD44^{hi}/CD62^{lo}) infiltrated rejecting kidney allografts. With this information, lists of human transcripts that can be used to identify activated/effector CD8 CTL within the leukocytic infiltrate observed in TCMR were produced using stringent criteria, such as a false discovery rate of 0.01 for transcripts with greater expression than in B cells, untreated and IFN-γ-treated monocytes, and nephrectomies, as well as exclusion of transcripts with a signal >200 in B cells, untreated and IFN treated monocytes, and nephrectomies.

Defining human CD8 CATs: Allo-stimulated human CTL were generated and transcript expression was examined by microarray analysis. An algorithm was used that selected for transcripts preferentially expressed (p<0.01) in human CD8 CTL compared to nephrectomy, B cells, and monocytes (FIG. 1). Since IFN-γ is abundantly produced in TCM and profoundly affects the kidney and monocytes/macrophages, IFN-γ-induced transcripts were selected against. Transcripts that were considered to be IFN-γ-induced transcripts were inducible more than 2-fold (p<0.05) in monocytes treated with IFN-γ. Such transcripts included CCL5; guanylate binding proteins GBP1, GBP2, and GBP5; interferon induced transmembrane protein 1 (IFITM1); and transporter associated with antigen processing 1 (TAP1) transcripts. Transcripts with an expression signal >200 in nephrectomy, B cells, and untreated or IFN-γ-treated monocytes were deleted from the list of transcripts. The resulting 205 CD8 CATs included transcripts for the cytolytic molecules granulysin (GNLY), granzyme B (GZMB), granzyme A (GZMA), granzyme H (GZMH), and granzyme K (GZMK); cell membrane receptors including CD8A/B; killer-cell lectin-like receptors (KLRK1/NKG2D, KLRD1/CD94, KLRC1/NKG2A, KLRC3/NKG2E, and KLRB1/NKR-P1); and T cell signaling polypeptides (LCK, ITK, CD3Z, CD3D, and RAC2; Table 2).

### Example 2

### Assessing CAT Expression in Human Kidney Transplants with TCMR

Human kidney transplant biopsies: Human kidney transplant biopsies (n=16) diagnosed as TCMR (T cell-mediated rejection) were selected from a patient cohort. The diagnosis of TCMR was based on histopathology using Banff criteria (Racusen et al., Kidney Int., 55:713-723 (1999)) and the clinical diagnosis of a rejection “episode” based on retrospective assessment of clinical course, independent of transcriptome analysis. Kidney tissues (n=8) from macroscopically and histologically unaffected areas of the cortex of native nephrectomies performed for renal carcinoma served as controls. In addition to the cores obtained for conventional diagnostic assessment, an 18-gauge biopsy core was collected for gene expression analysis (see Example 1), immediately placed in RNALater solution, kept at 4° C. for 4-24 hours, and then stored at −20° C. RNA was isolated as described in Example 1 above, and an average of 4 μg of RNA was obtained per core specimen.

CD8 CAT expression in index cases of TCMR: It was examined whether measurement of CAT expression could estimate CTL burden in kidney biopsies. The transcriptomes from TCMR biopsies were normalized and compared to nephrectomy samples. In mouse kidney transplants, the signals for the most highly expressed CATs were about 7-10 fold lower in kidneys with TCMR compared to CTL in vitro, presumably due to dilution by the mRNA from kidney and other genes (Einecke et al., Am. J. Transpl., 5:1827-1836 (2005)). Transcripts with higher expression in CTL than in normal kidney were detectable above the background of the chip after “dilution” in the transcripts from kidney and other inflammatory cells.

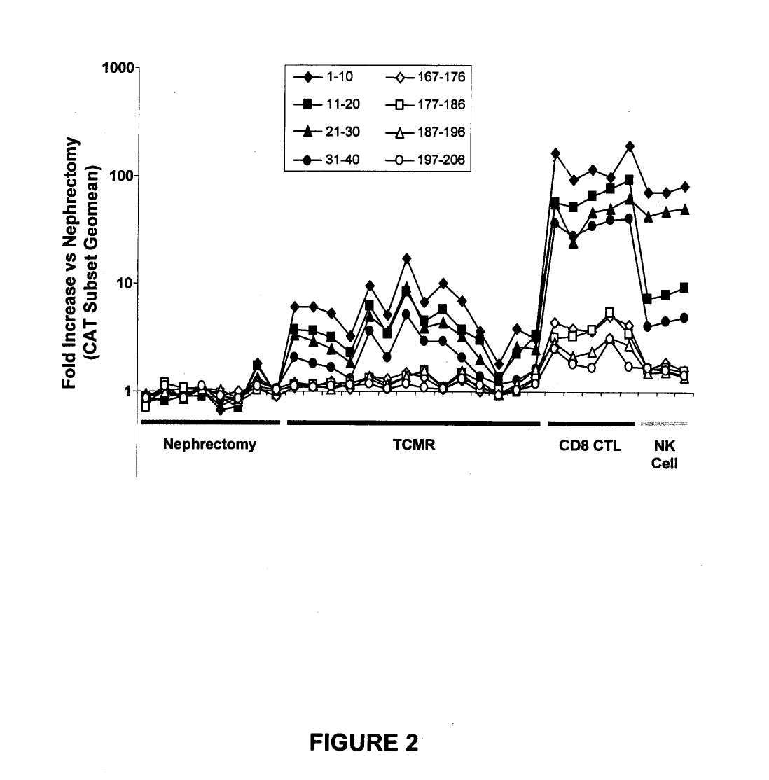

To determine whether this also occurred with CD8 CATs, the CD8 CATs were grouped into subsets of 10 transcripts, arranged by decreasing signal intensity in CD8 CTL. Expression of the top four subsets of CD8 CATs was higher in TCMR compared to nephrectomy and also differed between TCMR cases, likely representing differential CTL burden (FIG. 2). Quantitatively, the order of the CAT subsets was identical in each TCMR case, but on a variable absolute level reflecting different degrees of rejection. The order in which each subset was expressed in TCMR was also the same in which they are expressed in CD8 CTL in vitro. Four subsets of CATs that had the lowest expression in CTL in vitro were used as controls. These were not detectably elevated in the microarray result from human biopsies, because their signal faded into the background of the microarray system. Thus, only highly expressed CD8 CTL transcripts remain detectable after dilution in the inflammatory compartment of the tissue and can give consistent estimates of CTL burden in TCMR cases.

TABLE 1
CD8 CAT expression in nephrectomy, TCMR, treated TCMR, and ATN cases

| Affymetrix | | | GI | | StdErr | | StdErr |
|---|---|---|---|---|---|---|---|
| Probeset | Gene Symbol | Genbank | Number | Nephr | Norm | Signal | Signal |
| 205488_at | GZMA | NM_006144 | 6996012 | 0.9 | 0.2 | 24.9 | 5.8 |
| 210164_at | GZMB | J03189 | 338010 | 1.0 | 0.1 | 19.5 | 1.4 |
| 206666_at | GZMK | NM_002104 | 73747815 | 0.9 | 0.1 | 53.3 | 7.4 |
| 205831_at | CD2 | NM_001767 | 31542293 | 0.9 | 0.1 | 60.1 | 10.0 |
| 206366_x_at | XCL1 | U23772 | 902001 | 1.0 | 0.1 | 31.2 | 3.0 |
| 205758_at | CD8A | AW006735 | 5855513 | 1.0 | 0.1 | 62.9 | 8.3 |
| 231776_at | EOMES | NM_005442 | 22538469 | 1.0 | 0.1 | 27.0 | 2.7 |
| 204891_s_at | LCK | NM_005356 | 112789545 | 0.9 | 0.2 | 36.1 | 8.1 |
| 213915_at | NKG7 | NM_005601 | 49574503 | 1.0 | 0.1 | 30.0 | 1.9 |
| 205821_at | KLRK1 | NM_007360 | 6679051 | 1.0 | 0.1 | 64.3 | 7.8 |
| 213539_at | CD3D | NM_000732 | 98985799 | 1.0 | 0.1 | 98.0 | 7.2 |
| 207651_at | H963 | NM_013308 | 31377771 | 0.9 | 0.1 | 21.2 | 3.6 |
| 210031_at | CD3Z | J04132 | 623041 | 1.0 | 0.1 | 23.9 | 1.8 |
| 202503_s_at | KIAA0101 | NM_014736 | 71773764 | 0.9 | 0.2 | 36.8 | 7.1 |
| 211339_s_at | ITK | D13720 | 399657 | 1.0 | 0.1 | 16.5 | 2.2 |
| 210321_at | GZMH | M36118 | 181163 | 1.0 | 0.1 | 28.4 | 2.5 |
| 209671_x_at | TRA@ | M12423 | 338738 | 0.9 | 0.2 | 103.8 | 17.3 |
| 205291_at | IL2RB | NM_000878 | 23238195 | 0.9 | 0.1 | 77.5 | 6.4 |
| 37145_at | GNLY | M85276 | 189229 | 1.0 | 0.1 | 20.5 | 1.2 |
| 214617_at | PRF1 | AI445650 | 4289772 | 1.0 | 0.1 | 84.2 | 7.9 |
| 210140_at | CST7 | AF031824 | 3252857 | 1.0 | 0.1 | 50.4 | 5.1 |
| 216920_s_at | TRGV9 | M27331 | 540458 | 0.9 | 0.1 | 43.6 | 7.7 |
| 219118_at | FKBP11 | NM_016594 | 7706130 | 1.0 | 0.1 | 48.2 | 3.6 |
| 210354_at | IFNG | M29383 | 186514 | 1.0 | 0.1 | 17.7 | 1.6 |
| 219528_s_at | BCL11B | NM_022898 | 12597634 | 1.0 | 0.1 | 17.5 | 1.3 |
| 209773_s_at | RRM2 | BC001886 | 12804874 | 0.9 | 0.2 | 34.1 | 7.3 |
| 228273_at | FLJ11029 | BG165011 | 12671714 | 0.9 | 0.1 | 15.2 | 3.0 |
| 211597_s_at | HOP | AB059408 | 13560280 | 0.8 | 0.2 | 100.8 | 29.5 |
| 206118_at | STAT4 | NM_003151 | 21618332 | 1.0 | 0.1 | 35.1 | 4.2 |
| 1555613_a_at | ZAP70 | AB083211 | 26453339 | 1.0 | 0.1 | 29.5 | 2.4 |
| 218764_at | PRKCH | NM_024064 | 13129041 | 1.0 | 0.1 | 117.8 | 15.0 |
| 205456_at | CD3E | NM_000733 | 50726997 | 1.0 | 0.1 | 39.8 | 2.2 |
| 236782_at | SAMD3 | AI129628 | 3598142 | 1.0 | 0.1 | 13.7 | 0.7 |
| 200953_s_at | CCND2 | NM_001759 | 16950656 | 0.9 | 0.2 | 95.2 | 11.6 |
| 203213_at | CDC2 | AL524035 | 45699297 | 1.0 | 0.1 | 32.0 | 3.2 |
| 225841_at | FLJ30525 | BE502436 | 9704844 | 1.0 | 0.1 | 41.7 | 3.9 |
| 211005_at | LAT | AF036906 | 2828025 | 1.0 | 0.1 | 44.0 | 2.9 |
| 202746_at | ITM2A | AL021786 | 18650726 | 1.0 | 0.1 | 156.3 | 14.4 |
| 227295_at | IKIP | AW182575 | 6451035 | 1.0 | 0.1 | 31.4 | 3.1 |
| 228298_at | MGC16044 | BF056901 | 10810797 | 1.0 | 0.1 | 33.4 | 5.3 |
| 226771_at | ATP8B2 | AB032963 | 6329896 | 1.0 | 0.1 | 58.8 | 4.5 |
| 203362_s_at | MAD2L1 | NM_002358 | 6466452 | 1.0 | 0.1 | 16.3 | 1.5 |
| 204162_at | KNTC2 | NM_006101 | 5174456 | 1.0 | 0.1 | 12.9 | 2.1 |
| 228658_at | LOC150271 | R54042 | 815944 | 1.0 | 0.1 | 13.7 | 0.3 |
| 206974_at | CXCR6 | NM_006564 | 5730105 | 1.0 | 0.1 | 30.3 | 2.1 |
| 219423_x_at | TNFRSF25 | NM_003790 | 23200019 | 1.0 | 0.0 | 25.5 | 3.0 |
| 214710_s_at | CCNB1 | BE407516 | 9343966 | 1.0 | 0.1 | 23.5 | 1.6 |
| 203413_at | NELL2 | NM_006159 | 5453765 | 1.0 | 0.1 | 56.2 | 7.2 |
| 218039_at | NUSAP1 | NM_016359 | 59710089 | 0.9 | 0.2 | 51.5 | 7.0 |
| 204825_at | MELK | NM_014791 | 41281490 | 1.0 | 0.1 | 30.2 | 2.8 |
| 203675_at | NUCB2 | NM_005013 | 116063564 | 0.9 | 0.2 | 117.0 | 15.9 |
| 224851_at | CDK6 | AW274756 | 6661786 | 1.0 | 0.1 | 64.8 | 7.2 |
| 235735_at | TNFSF8 | AI936516 | 5675386 | 1.0 | 0.1 | 16.3 | 1.2 |
| 218009_s_at | PRC1 | NM_003981 | 40807441 | 1.0 | 0.1 | 16.2 | 1.7 |
| 217147_s_at | TRIM | AJ240085 | 6911580 | 1.0 | 0.1 | 10.5 | 0.6 |
| 207681_at | CXCR3 | NM_001504 | 4504098 | 0.9 | 0.1 | 48.8 | 5.8 |
| 1555758_a_at | CDKN3 | AF213040 | 12734657 | 1.0 | 0.1 | 15.7 | 1.5 |
| 211210_x_at | SH2D1A | AF100539 | 3928893 | 1.0 | 0.1 | 41.1 | 2.3 |
| 213958_at | CD6 | AW134823 | 6138369 | 0.9 | 0.1 | 107.5 | 9.3 |
| 220485_s_at | SIRPB2 | NM_018556 | 94538334 | 1.0 | 0.1 | 21.0 | 3.4 |
| 240070_at | FLJ39873 | AW512550 | 7150628 | 1.0 | 0.1 | 12.5 | 0.6 |
| 220418_at | UBASH3A | NM_018961 | 49640012 | 1.0 | 0.1 | 15.7 | 1.1 |
| 203358_s_at | EZH2 | NM_004456 | 23510382 | 1.0 | 0.1 | 12.7 | 1.3 |
| 1558972_s_at | C6orf190 | BC043608 | 27694410 | 1.0 | 0.1 | 8.2 | 0.6 |
| 201292_at | TOP2A | AL561834 | 46225618 | 1.0 | 0.1 | 11.4 | 1.6 |
| 206632_s_at | APOBEC3B | NM_004900 | 22907024 | 1.0 | 0.1 | 12.7 | 1.2 |
| 222077_s_at | RACGAP1 | AU153848 | 11015369 | 0.9 | 0.2 | 75.5 | 9.3 |
| 204552_at | INPP4A | AA355179 | 2007571 | 0.9 | 0.1 | 43.0 | 8.2 |
| 224428_s_at | CDCA7 | AY029179 | 13641303 | 0.9 | 0.1 | 17.8 | 2.6 |
| 222786_at | CHST12 | BC002918 | 12804130 | 1.0 | 0.1 | 64.3 | 5.2 |
| 214450_at | CTSW | NM_001335 | 119395730 | 1.0 | 0.1 | 58.8 | 6.7 |
| 202954_at | UBE2C | NM_007019 | 32967292 | 1.0 | 0.1 | 40.2 | 3.3 |
| 222920_s_at | KIAA0748 | B0231515 | 12726617 | 0.9 | 0.1 | 26.0 | 2.5 |
| 230337_at | SOS1 | AW241962 | 6575716 | 1.0 | 0.1 | 85.5 | 13.4 |
| 203755_at | BUB1B | NM_001211 | 59814246 | 1.0 | 0.1 | 15.0 | 1.9 |
| 202705_at | CCNB2 | NM_004701 | 10938017 | 1.0 | 0.1 | 26.8 | 1.8 |
| 208807_s_at | CHD3 | U91543 | 3298561 | 1.0 | 0.1 | 127.3 | 16.5 |
| 219479_at | KDELC1 | NM_024089 | 13129085 | 1.0 | 0.1 | 40.1 | 5.9 |
| 210439_at | ICOS | AB023135 | 5360718 | 1.0 | 0.1 | 14.0 | 1.0 |
| 204256_at | ELOVL6 | NM_024090 | 13129087 | 1.0 | 0.1 | 30.5 | 2.8 |
| 229261_at | ITGA3 | BF4508819 | 11592117 | 1.0 | 0.1 | 36.1 | 5.1 |
| 232234_at | SLA2 | AA305476 | 1957823 | 1.0 | 0.1 | 51.5 | 4.3 |
| 218662_s_at | HCAP-G | NM_022346 | 50658080 | 1.0 | 0.1 | 15.1 | 1.1 |
| 219918_s_at | ASPM | NM_018123 | 8922484 | 1.0 | 0.1 | 8.9 | 0.6 |
| 209408_at | KIF2C | U63743 | 1695881 | 1.0 | 0.1 | 34.7 | 3.3 |
| 226876_at | MGC45871 | AI961778 | 5754491 | 0.9 | 0.1 | 62.9 | 6.2 |
| 207072_at | IL18RAP | NM_003853 | 27477087 | 1.0 | 0.1 | 29.5 | 2.5 |
| 40472_at | LOC254531 | AF007155 | 2852635 | 1.0 | 0.1 | 51.9 | 2.3 |
| 212136_at | ATP2B4 | AW517686 | 7155768 | 1.0 | 0.1 | 95.6 | 13.3 |
| 218086_at | NPDC1 | NM_015392 | 20149616 | 1.0 | 0.1 | 108.5 | 11.2 |
| 227212_s_at | PHF19 | BE544837 | 9773482 | 1.0 | 0.1 | 37.8 | 4.7 |
| 206486_at | LAG3 | NM_002286 | 15718681 | 1.0 | 0.1 | 19.0 | 2.2 |
| 206761_at | CD96 | NM_005816 | 93141043 | 1.0 | 0.1 | 11.4 | 0.7 |
| 202870_s_at | CDC20 | NM_001255 | 118402581 | 0.9 | 0.1 | 24.7 | 2.1 |
| 228280_at | MGC14289 | AI188445 | 3739654 | 1.0 | 0.1 | 131.0 | 14.3 |
| 207165_at | HMMR | NM_012485 | 7108350 | 1.0 | 0.1 | 19.3 | 2.1 |
| 207351_s_at | SH2D2A | NM_003975 | 31543620 | 1.0 | 0.1 | 49.0 | 3.6 |
| 210038_at | PRKCQ | AL137145 | 9581557 | 1.0 | 0.1 | 103.7 | 9.5 |
| 203989_x_at | F2R | NM_001992 | 6031164 | 0.9 | 0.2 | 109.3 | 20.8 |
| 230489_at | CD5 | AI797836 | 5363393 | 1.0 | 0.1 | 43.0 | 2.7 |
| 1562697_at | LOC339988 | BC041468 | 27370762 | 1.0 | 0.0 | 48.7 | 2.6 |
| 207979_s_at | CD8B1 | NM_004931 | 90421320 | 1.0 | 0.1 | 46.3 | 5.1 |
| 207604_s_at | SLC4A7 | NM_003615 | 134288864 | 0.9 | 0.1 | 36.6 | 6.0 |
| 210052_s_at | TPX2 | AF098158 | 6073830 | 1.0 | 0.1 | 24.0 | 1.9 |
| 202789_at | PLCG1 | AL022394 | 11345540 | 1.0 | 0.0 | 109.6 | 7.1 |
| 219566_at | PLEKHF1 | NM_024310 | 141803009 | 1.0 | 0.1 | 84.8 | 9.6 |
| 202095_s_at | BIRC5 | NM_001168 | 59859877 | 1.0 | 0.1 | 20.8 | 1.0 |
| 202589_at | TYMS | NM_001071 | 4507750 | 0.9 | 0.1 | 200.3 | 23.6 |
| 220307_at | CD244 | NM_016382 | 51593100 | 1.0 | 0.1 | 34.4 | 2.6 |
| 218755_at | KIF20A | NM_005733 | 5032012 | 1.0 | 0.1 | 15.9 | 1.6 |
| 227747_at | LOC196264 | AA772172 | 2823955 | 1.0 | 0.1 | 31.2 | 4.4 |
| 210865_at | TNFSF6 | D38122 | 601892 | 1.0 | 0.1 | 36.6 | 2.6 |
| 1552307_a_at | C18orf17 | NM_153211 | 116256461 | 1.0 | 0.1 | 18.7 | 1.1 |
| 223307_at | CDCA3 | BC002551 | 33876851 | 1.0 | 0.1 | 21.5 | 1.4 |
| 206785_s_at | KLRC1 | NM_002260 | 75709168 | 1.0 | 0.1 | 9.1 | 0.5 |
| 227915_at | ASB2 | AI872284 | 5546333 | 1.0 | 0.1 | 56.1 | 6.1 |
| 238600_at | MARLIN1 | AW157571 | 6228972 | 1.0 | 0.1 | 16.2 | 1.1 |
| 238423_at | SYTL3 | AI674404 | 4874884 | 1.0 | 0.1 | 71.4 | 8.8 |
| 236341_at | CTLA4 | AI733018 | 5054131 | 1.0 | 0.1 | 11.1 | 0.9 |
| 204015_s_at | DUSP4 | BC002671 | 33877002 | 1.0 | 0.1 | 35.5 | 3.4 |
| 234970_at | MTAC2D1 | AI741469 | 5109757 | 1.0 | 0.1 | 25.8 | 2.6 |
| 223229_at | HSPC150 | AB032931 | 7416119 | 1.0 | 0.1 | 33.4 | 3.7 |
| 205005_s_at | NMT2 | AW293531 | 6700167 | 0.9 | 0.1 | 69.1 | 6.5 |
| 228722_at | HRMT1L1 | AI928367 | 5664331 | 1.0 | 0.1 | 74.0 | 6.8 |
| 203764_at | DLG7 | NM_014750 | 21361644 | 1.0 | 0.1 | 11.7 | 0.9 |
| 234973_at | SLC38A5 | BG325630 | 13132067 | 1.0 | 0.0 | 44.0 | 3.9 |
| 206980_s_at | FLT3LG | NM_001459 | 38455415 | 1.0 | 0.1 | 36.0 | 3.5 |
| 214551_s_at | CD7 | NM_006137 | 61676094 | 1.0 | 0.1 | 18.8 | 1.0 |
| 220684_at | TBX21 | NM_013351 | 7019548 | 1.0 | 0.1 | 38.1 | 2.9 |
| 204917_s_at | MLLT3 | AV756536 | 55952930 | 1.0 | 0.1 | 76.9 | 5.1 |
| 205790_at | SCAP1 | NM_003726 | 115527073 | 1.0 | 0.1 | 110.8 | 9.6 |
| 218585_s_at | RAMP | NM_016448 | 7705575 | 1.0 | 0.1 | 10.7 | 1.1 |
| 34726_at | CACNB3 | U07139 | 463890 | 1.0 | 0.1 | 28.2 | 2.7 |
| 204962_s_at | CENPA | NM_001809 | 109637780 | 1.0 | 0.1 | 13.3 | 0.6 |
| 1558102_at | TM6SF1 | AK055438 | 16550163 | 1.0 | 0.1 | 24.3 | 2.1 |
| 206589_at | GFI1 | NM_005263 | 71037376 | 1.0 | 0.1 | 28.3 | 2.3 |
| 230536_at | PBX4 | AJ300182 | 12697317 | 1.0 | 0.1 | 32.5 | 4.6 |
| 226299_at | PKN3 | NM_013355 | 42516554 | 1.0 | 0.1 | 43.7 | 4.3 |
| 1558304_s_at | LOC283989 | BI905836 | 16168427 | 1.0 | 0.0 | 89.1 | 5.8 |
| 212772_s_at | ABCA2 | AL162060 | 7328110 | 1.0 | 0.1 | 87.4 | 7.6 |
| 206804_at | CD3G | NM_000073 | 4557428 | 1.0 | 0.1 | 30.6 | 4.3 |
| 212022_s_at | MKI67 | BF001806 | 10702081 | 1.0 | 0.1 | 26.7 | 1.8 |
| 203418_at | CCNA2 | NM_001237 | 16950653 | 1.0 | 0.1 | 16.0 | 1.6 |
| 235476_at | TSBF1 | AW182459 | 6450919 | 1.0 | 0.1 | 16.2 | 1.0 |
| 1554539_a_at | RHOF | BC018208 | 17390467 | 1.0 | 0.1 | 30.9 | 3.4 |
| 209840_s_at | LRRN3 | AI221950 | 3804153 | 1.0 | 0.1 | 20.6 | 1.1 |
| 227868_at | LOC154761 | AI928764 | 5664728 | 1.0 | 0.1 | 50.1 | 4.0 |
| 219315_s_at | FLJ20898 | NM_024600 | 142376826 | 1.0 | 0.1 | 153.2 | 7.3 |
| 229268_at | RABEP1 | AA723152 | 2740929 | 0.9 | 0.1 | 76.2 | 17.4 |
| 216693_x_at | HDGFRP3 | AL133102 | 6453585 | 1.0 | 0.1 | 100.2 | 9.4 |
| 209642_at | BUB1 | AF043294 | 6970210 | 1.0 | 0.1 | 8.7 | 0.7 |
| 222039_at | LOC146909 | AA292789 | 1941611 | 1.0 | 0.1 | 29.0 | 2.2 |
| 226661_at | CDCA2 | T90295 | 718808 | 1.0 | 0.1 | 13.7 | 0.8 |
| 238846_at | TNFRSF11A | AW026379 | 5879909 | 1.0 | 0.1 | 9.8 | 0.7 |
| 235816_s_at | Rgr | AI867408 | 5540424 | 1.0 | 0.1 | 16.3 | 0.7 |
| 227313_at | MGC40499 | AI870866 | 5544834 | 1.0 | 0.0 | 150.4 | 12.9 |
| 207183_at | GPR19 | NM_006143 | 5453665 | 1.0 | 0.1 | 17.0 | 1.0 |
| 207315_at | CD226 | NM_006566 | 5729807 | 1.0 | 0.1 | 10.8 | 0.3 |
| 1553423_a_at | FLJ31952 | NM_144682 | 142357995 | 1.0 | 0.1 | 8.9 | 0.4 |
| 206039_at | RAB33A | NM_004794 | 34485717 | 1.0 | 0.1 | 13.0 | 1.2 |
| 206999_at | IL12RB2 | NM_001559 | 24497441 | 1.0 | 0.1 | 11.6 | 1.0 |
| 207723_s_at | KLRC3 | NM_002261 | 75709174 | 1.0 | 0.1 | 8.2 | 0.7 |
| 208511_at | PTTG2 | NM_021000 | 10337606 | 1.0 | 0.1 | 12.8 | 1.0 |
| 210837_s_at | PDE4D | AF012074 | 2735858 | 1.0 | 0.1 | 12.4 | 0.8 |
| 211207_s_at | ACSL6 | AF129166 | 5702201 | 1.0 | 0.1 | 11.7 | 0.7 |
| 211861_x_at | CD28 | AF222343 | 6969516 | 1.0 | 0.1 | 8.8 | 1.0 |
| 235266_at | ATAD2 | AI139629 | 3645601 | 1.0 | 0.1 | 11.3 | 0.5 |
| 241871_at | CAMK4 | AL529104 | 31066950 | 1.0 | 0.1 | 8.3 | 0.5 |
| 225387_at | TM4SF9 | AA059445 | 1553278 | 1.0 | 0.1 | 53.5 | 3.9 |
| 209464_at | AURKB | AB011446 | 5688865 | 1.0 | 0.1 | 16.8 | 1.7 |
| 204040_at | RNF144 | NM_014746 | 38045937 | 1.0 | 0.1 | 92.6 | 9.5 |
| 206506_s_at | SUPT3H | NM_003599 | 141802388 | 1.0 | 0.1 | 64.1 | 7.9 |
| 215797_at | MGC40069 | AE000659 | 2358025 | 1.0 | 0.1 | 15.0 | 1.6 |
| 207460_at | GZMM | NM_005317 | 7108347 | 1.0 | 0.1 | 37.4 | 5.0 |
| 221582_at | HIST3H2A | BC001193 | 12654706 | 1.0 | 0.0 | 89.9 | 5.9 |
| 227686_at | MGC15763 | BE465433 | 9511208 | 1.0 | 0.1 | 153.4 | 15.7 |
| 218726_at | DKFZp762E1312 | NM_018410 | 83816963 | 1.0 | 0.1 | 25.8 | 2.5 |
| 212728_at | DLG3 | T62872 | 666529 | 1.0 | 0.1 | 105.0 | 14.5 |
| 236717_at | LOC165186 | AI632621 | 4683951 | 1.0 | 0.0 | 35.8 | 3.4 |
| 222777_s_at | WHSC1 | AI770166 | 5236621 | 1.0 | 0.1 | 23.4 | 2.0 |
| 206518_s_at | RGS9 | NM_003835 | 126365760 | 1.0 | 0.1 | 20.5 | 1.2 |
| 209197_at | SYT11 | AA626780 | 2539167 | 1.0 | 0.1 | 51.1 | 6.2 |
| 212949_at | BRRN1 | D38553 | 559714 | 1.0 | 0.1 | 27.8 | 2.2 |
| 213623_at | KIF3A | NM_007054 | 83716022 | 1.0 | 0.1 | 29.0 | 2.6 |
| 208524_at | GPR15 | NM_005290 | 4885298 | 1.0 | 0.1 | 20.3 | 1.6 |
| 218434_s_at | AACS | NM_023928 | 111154071 | 1.0 | 0.0 | 151.2 | 9.3 |
| 202179_at | BLMH | NM_000386 | 33591068 | 1.0 | 0.1 | 151.3 | 8.7 |
| 209602_s_at | GATA3 | AI796169 | 5361632 | 0.9 | 0.1 | 70.9 | 4.6 |
| 204159_at | CDKN2C | NM_001262 | 17981697 | 1.0 | 0.1 | 128.5 | 18.0 |
| 226627_at | SEPT8 | AI912094 | 5631949 | 1.0 | 0.0 | 61.2 | 3.9 |
| 224832_at | DUSP16 | AB051487 | 12697944 | 1.0 | 0.1 | 149.4 | 9.8 |
| 220576_at | PGAP1 | NM_024989 | 62865856 | 1.0 | 0.0 | 20.6 | 3.2 |
| 220613_s_at | SYTL2 | NM_017695 | 8923162 | 1.0 | 0.0 | 34.7 | 3.4 |
| 210763_x_at | NCR3 | AF031137 | 2623874 | 1.0 | 0.1 | 59.0 | 6.7 |
| 203565_s_at | MNAT1 | NM_002431 | 49574512 | 1.0 | 0.1 | 88.5 | 7.0 |
| 225842_at | PHLDA1 | AK026181 | 10438947 | 1.0 | 0.1 | 158.0 | 24.7 |
| 214950_at | IL9R | L39064 | 632992 | 1.0 | 0.1 | 56.2 | 6.7 |
| 220941_s_at | C21orf91 | NM_017447 | 40254881 | 1.0 | 0.1 | 41.9 | 5.8 |
| 208406_s_at | GRAP2 | NM_004810 | 19913386 | 1.0 | 0.1 | 48.5 | 4.8 |
| 215195_at | PRKCA | AF035594 | 3168857 | 1.0 | 0.1 | 65.2 | 5.8 |
| 211685_s_at | NCALD | AF251061 | 13625183 | 1.0 | 0.1 | 143.7 | 14.5 |
| 205171_at | PTPN4 | NM_002830 | 18104987 | 1.0 | 0.1 | 117.9 | 8.6 |
| 226433_at | RNF157 | BF056204 | 10810090 | 1.0 | 0.1 | 74.1 | 8.0 |
| 227678_at | KUB3 | AI628122 | 4664922 | 0.9 | 0.2 | 62.0 | 9.4 |
| 210426_x_at | RORA | U04897 | 451563 | 0.9 | 0.1 | 37.8 | 5.9 |

| Affymetrix | | StdErr | Treated | StdErr | | StdErr | CD8 | StdErr |
|---|---|---|---|---|---|---|---|---|
| Probeset | TCMR | Norm | TCMR | Norm | ATN | Norm | CTL | Norm |
| 205488_at | 7.8 | 1.8 | 3.6 | 1.3 | 1.5 | 0.2 | 286.6 | 29.2 |
| 210164_at | 7.3 | 1.8 | 2.0 | 0.7 | 1.3 | 0.1 | 260.6 | 37.2 |
| 206666_at | 7.2 | 1.3 | 2.8 | 0.9 | 1.4 | 0.2 | 49.6 | 18.0 |
| 205831_at | 6.5 | 1.2 | 2.2 | 0.2 | 1.7 | 0.2 | 76.1 | 11.7 |
| 206366_x_at | 6.0 | 1.3 | 2.3 | 0.6 | 1.2 | 0.1 | 56.6 | 11.2 |
| 205758_at | 5.9 | 1.1 | 2.5 | 0.2 | 1.2 | 0.1 | 103.3 | 9.4 |
| 231776_at | 5.8 | 1.0 | 2.6 | 0.3 | 1.1 | 0.1 | 48.9 | 15.5 |
| 204891_s_at | 5.8 | 1.2 | 1.8 | 0.1 | 1.5 | 0.2 | 121.0 | 5.4 |
| 213915_at | 4.9 | 0.9 | 2.4 | 0.4 | 1.7 | 0.2 | 129.6 | 12.2 |
| 205821_at | 4.9 | 0.6 | 2.6 | 0.7 | 1.7 | 0.1 | 66.1 | 19.8 |
| 213539_at | 4.9 | 0.9 | 1.7 | 0.0 | 1.3 | 0.1 | 68.6 | 4.5 |
| 207651_at | 4.7 | 1.1 | 1.8 | 0.0 | 1.1 | 0.1 | 94.4 | 16.8 |
| 210031_at | 4.6 | 0.6 | 2.0 | 0.0 | 1.7 | 0.2 | 69.9 | 7.2 |
| 202503_s_at | 4.5 | 0.7 | 2.5 | 0.4 | 2.8 | 0.5 | 58.6 | 23.8 |
| 211339_s_at | 4.4 | 1.0 | 1.3 | 0.2 | 1.2 | 0.1 | 79.9 | 18.8 |
| 210321_at | 4.4 | 0.9 | 2.1 | 1.0 | 1.3 | 0.2 | 59.6 | 10.4 |
| 209671_x_at | 4.1 | 0.6 | 1.8 | 0.2 | 1.4 | 0.1 | 63.1 | 4.9 |
| 205291_at | 3.7 | 0.6 | 1.8 | 0.1 | 1.4 | 0.1 | 53.7 | 5.6 |
| 37145_at | 3.7 | 0.8 | 2.1 | 0.7 | 1.7 | 0.3 | 545.5 | 113.5 |
| 214617_at | 3.6 | 0.7 | 1.9 | 0.4 | 1.4 | 0.1 | 72.1 | 12.3 |
| 210140_at | 3.6 | 0.4 | 1.9 | 0.2 | 1.1 | 0.1 | 69.4 | 14.3 |
| 216920_s_at | 3.6 | 0.7 | 1.2 | 0.1 | 1.2 | 0.2 | 32.8 | 11.2 |
| 219118_at | 3.5 | 0.9 | 1.6 | 0.2 | 1.5 | 0.2 | 15.2 | 3.5 |
| 210354_at | 3.3 | 0.8 | 1.5 | 0.1 | 1.1 | 0.0 | 66.1 | 55.8 |
| 219528_s_at | 3.2 | 0.7 | 1.6 | 0.2 | 1.2 | 0.1 | 36.8 | 11.5 |
| 209773_s_at | 3.1 | 0.7 | 2.0 | 0.2 | 1.9 | 0.4 | 70.7 | 39.9 |
| 228273_at | 2.9 | 0.6 | 1.3 | 0.1 | 1.5 | 0.2 | 32.4 | 12.8 |
| 211597_s_at | 2.9 | 0.6 | 1.2 | 0.0 | 2.1 | 0.8 | 57.0 | 7.2 |
| 206118_at | 2.8 | 0.4 | 1.1 | 0.1 | 1.4 | 0.1 | 22.4 | 6.4 |
| 1555613_a_at | 2.8 | 0.6 | 1.2 | 0.2 | 1.2 | 0.1 | 43.8 | 7.8 |
| 218764_at | 2.8 | 0.3 | 1.4 | 0.2 | 1.4 | 0.1 | 19.0 | 2.6 |
| 205456_at | 2.6 | 0.4 | 1.2 | 0.1 | 1.3 | 0.1 | 38.0 | 4.4 |
| 236782_at | 2.5 | 0.3 | 1.6 | 0.3 | 1.2 | 0.0 | 27.2 | 42.8 |
| 200953_s_at | 2.5 | 0.5 | 1.2 | 0.1 | 2.0 | 0.4 | 18.7 | 3.8 |
| 203213_at | 2.5 | 0.3 | 1.9 | 0.1 | 2.1 | 0.3 | 21.1 | 11.5 |
| 225841_at | 2.4 | 0.2 | 1.6 | 0.4 | 1.5 | 0.1 | 15.9 | 3.8 |
| 211005_at | 2.4 | 0.4 | 1.1 | 0.2 | 1.2 | 0.1 | 27.5 | 2.3 |
| 202746_at | 2.4 | 0.4 | 1.6 | 0.4 | 1.2 | 0.1 | 8.8 | 1.8 |
| 227295_at | 23 | 0.3 | 1.5 | 0.1 | 2.1 | 0.3 | 12.0 | 1.8 |
| 228298_at | 2.3 | 0.3 | 0.9 | 0.1 | 1.0 | 0.1 | 14.8 | 2.5 |
| 226771_at | 2.3 | 0.4 | 1.3 | 0.1 | 1.3 | 0.1 | 9.0 | 1.1 |
| 203362_s_at | 2.2 | 0.3 | 1.8 | 0.3 | 2.0 | 0.2 | 30.2 | 12.6 |
| 204162_at | 2.2 | 0.3 | 1.2 | 0.0 | 1.2 | 0.0 | 33.6 | 12.9 |
| 228658_at | 2.2 | 0.4 | 1.2 | 0.1 | 1.2 | 0.0 | 57.1 | 17.4 |
| 206974_at | 2.1 | 0.3 | 1.5 | 0.1 | 0.8 | 0.0 | 49.9 | 12.7 |
| 219423_x_at | 2.1 | 0.4 | 1.1 | 0.1 | 1.5 | 0.2 | 8.8 | 1.3 |
| 214710_s_at | 2.1 | 0.3 | 1.9 | 0.1 | 2.3 | 0.3 | 17.0 | 8.6 |
| 203413_at | 2.0 | 0.3 | 0.8 | 0.1 | 0.9 | 0.1 | 19.0 | 8.5 |
| 218039_at | 2.0 | 0.3 | 1.2 | 0.1 | 1.7 | 0.2 | 18.2 | 6.5 |
| 204825_at | 2.0 | 0.3 | 1.4 | 0.1 | 1.3 | 0.1 | 15.0 | 7.3 |
| 203675_at | 2.0 | 0.3 | 1.5 | 0.2 | 1.8 | 0.1 | 4.2 | 0.6 |
| 224851_at | 1.9 | 0.2 | 1.1 | 0.1 | 1.9 | 0.2 | 7.5 | 2.7 |
| 235735_at | 1.9 | 0.3 | 1.3 | 0.0 | 1.2 | 0.0 | 12.0 | 1.8 |
| 218009_s_at | 1.9 | 0.2 | 1.3 | 0.2 | 1.8 | 0.3 | 21.2 | 7.4 |
| 217147_s_at | 1.9 | 0.4 | 1.2 | 0.1 | 1.2 | 0.0 | 12.8 | 7.7 |
| 207681_at | 1.9 | 0.3 | 1.1 | 0.1 | 0.6 | 0.0 | 31.9 | 6.9 |
| 1555758_a_at | 1.9 | 0.2 | 1.8 | 0.3 | 1.4 | 0.1 | 23.3 | 7.6 |
| 211210_x_at | 1.9 | 0.1 | 1.3 | 0.2 | 0.8 | 0.1 | 26.7 | 7.4 |
| 213958_at | 1.9 | 0.2 | 1.3 | 0.0 | 0.8 | 0.0 | 7.2 | 0.7 |
| 220485_s_at | 1.9 | 0.3 | 1.1 | 0.0 | 1.0 | 0.0 | 35.3 | 6.3 |
| 240070_at | 1.9 | 0.3 | 1.2 | 0.1 | 1.2 | 0.0 | 12.0 | 5.1 |
| 220418_at | 1.8 | 0.3 | 1.2 | 0.1 | 1.2 | 0.0 | 22.1 | 4.5 |
| 203358_s_at | 1.8 | 0.3 | 1.2 | 0.1 | 1.3 | 0.1 | 17.6 | 5.9 |
| 1558972_s_at | 1.8 | 0.3 | 1.2 | 0.1 | 1.2 | 0.0 | 48.2 | 6.9 |
| 201292_at | 1.8 | 0.3 | 1.2 | 0.0 | 1.2 | 0.0 | 26.6 | 11.5 |
| 206632_s_at | 1.8 | 0.3 | 1.3 | 0.1 | 1.8 | 0.3 | 15.2 | 5.5 |
| 222077_s_at | 1.8 | 0.2 | 1.6 | 0.2 | 1.3 | 0.1 | 12.0 | 3.9 |
| 204552_at | 1.8 | 0.2 | 1.1 | 0.2 | 1.5 | 0.1 | 9.0 | 1.5 |
| 224428_s_at | 1.8 | 0.3 | 1.2 | 0.2 | 1.1 | 0.0 | 42.0 | 9.2 |
| 222786_at | 1.7 | 0.1 | 1.0 | 0.1 | 1.4 | 0.1 | 5.9 | 1.0 |
| 214450_at | 1.7 | 0.2 | 0.9 | 0.1 | 1.1 | 0.1 | 43.7 | 3.7 |
| 202954_at | 1.7 | 0.2 | 1.5 | 0.3 | 1.2 | 0.1 | 22.1 | 10.4 |
| 222920_s_at | 1.7 | 0.2 | 1.2 | 0.1 | 0.9 | 0.0 | 19.6 | 6.6 |
| 230337_at | 1.7 | 0.2 | 1.5 | 0.5 | 1.6 | 0.2 | 5.4 | 1.2 |
| 203755_at | 1.7 | 0.2 | 1.4 | 0.1 | 1.2 | 0.1 | 22.4 | 9.9 |
| 202705_at | 1.7 | 0.2 | 1.6 | 0.2 | 1.3 | 0.1 | 27.3 | 9.9 |
| 208807_s_at | 1.7 | 0.2 | 1.4 | 0.2 | 1.6 | 0.2 | 3.2 | 0.3 |
| 219479_at | 1.7 | 0.1 | 1.5 | 0.1 | 2.1 | 0.3 | 4.8 | 0.9 |
| 210439_at | 1.6 | 0.3 | 1.2 | 0.1 | 1.2 | 0.0 | 20.8 | 12.1 |
| 204256_at | 1.6 | 0.1 | 1.9 | 0.2 | 2.4 | 0.3 | 3.5 | 1.3 |
| 229261_at | 1.6 | 0.2 | 1.2 | 0.2 | 1.5 | 0.2 | 5.2 | 1.5 |
| 232234_at | 1.6 | 0.1 | 1.2 | 0.1 | 0.8 | 0.0 | 19.6 | 1.1 |
| 218662_s_at | 1.6 | 0.2 | 1.2 | 0.1 | 1.2 | 0.0 | 12.6 | 5.9 |
| 219918_s_at | 1.6 | 0.2 | 1.2 | 0.1 | 1.2 | 0.0 | 16.8 | 13.3 |
| 209408_at | 1.6 | 0.1 | 1.2 | 0.0 | 1.1 | 0.1 | 21.1 | 7.8 |
| 226876_at | 1.6 | 0.1 | 1.3 | 0.1 | 1.5 | 0.1 | 17.0 | 4.0 |
| 207072_at | 1.6 | 0.3 | 1.2 | 0.2 | 0.9 | 0.0 | 11.8 | 4.1 |
| 40472_at | 1.6 | 0.1 | 1.1 | 0.1 | 1.4 | 0.1 | 7.0 | 1.3 |
| 212136_at | 1.6 | 0.2 | 1.2 | 0.1 | 1.5 | 0.1 | 5.6 | 1.2 |
| 218086_at | 1.6 | 0.2 | 1.1 | 0.1 | 1.4 | 0.2 | 2.1 | 0.2 |
| 227212_s_at | 1.6 | 0.1 | 1.0 | 0.2 | 1.2 | 0.1 | 19.2 | 6.2 |
| 206486_at | 1.6 | 0.2 | 1.1 | 0.1 | 1.1 | 0.0 | 44.0 | 9.0 |
| 206761_at | 1.6 | 0.2 | 1.2 | 0.1 | 1.2 | 0.0 | 35.4 | 16.7 |
| 202870_s_at | 1.6 | 0.2 | 1.5 | 0.3 | 1.5 | 0.2 | 20.9 | 11.3 |
| 228280_at | 1.6 | 0.2 | 1.7 | 0.0 | 2.0 | 0.3 | 3.1 | 1.0 |
| 207165_at | 1.6 | 0.2 | 1.5 | 0.3 | 1.2 | 0.1 | 13.3 | 8.3 |
| 207351_s_at | 1.5 | 0.2 | 0.9 | 0.1 | 0.9 | 0.0 | 18.2 | 1.9 |
| 210038_at | 1.5 | 0.2 | 1.0 | 0.4 | 1.7 | 0.2 | 10.3 | 1.1 |
| 203989_x_at | 1.5 | 0.2 | 1.1 | 0.1 | 1.4 | 0.2 | 2.0 | 0.8 |
| 230489_at | 1.5 | 0.1 | 1.2 | 0.2 | 0.9 | 0.1 | 14.2 | 2.1 |
| 1562697_at | 1.5 | 0.1 | 1.3 | 0.1 | 1.0 | 0.0 | 8.8 | 2.9 |
| 207979_s_at | 1.5 | 0.1 | 0.9 | 0.0 | 0.8 | 0.1 | 34.4 | 4.6 |
| 207604_s_at | 1.5 | 0.2 | 1.3 | 0.3 | 2.3 | 0.3 | 6.2 | 0.8 |
| 210052_s_at | 1.5 | 0.2 | 1.2 | 0.1 | 1.1 | 0.1 | 23.3 | 9.2 |
| 202789_at | 1.5 | 0.1 | 1.4 | 0.1 | 1.5 | 0.1 | 5.2 | 1.0 |
| 219566_at | 1.5 | 0.1 | 1.1 | 0.1 | 0.9 | 0.0 | 6.7 | 0.9 |
| 202095_s_at | 1.5 | 0.1 | 1.2 | 0.1 | 1.3 | 0.1 | 16.1 | 8.6 |
| 202589_at | 1.5 | 0.2 | 1.2 | 0.1 | 1.6 | 0.1 | 10.7 | 4.5 |
| 220307_at | 1.4 | 0.1 | 1.1 | 0.0 | 1.0 | 0.0 | 7.0 | 0.6 |
| 218755_at | 1.4 | 0.1 | 1.2 | 0.1 | 1.3 | 0.1 | 13.7 | 5.9 |
| 227747_at | 1.4 | 0.2 | 1.2 | 0.1 | 1.4 | 0.1 | 16.2 | 8.1 |
| 210865_at | 1.4 | 0.1 | 1.2 | 0.1 | 0.7 | 0.0 | 15.21 | 3.1 |
| 1552307_a_at | 1.4 | 0.1 | 1.2 | 0.1 | 1.2 | 0.0 | 11.8 | 1.1 |
| 223307_at | 1.4 | 0.1 | 1.4 | 0.2 | 1.2 | 0.1 | 12.9 | 5.5 |
| 206785_s_at | 1.4 | 0.1 | 1.2 | 0.1 | 1.2 | 0.0 | 17.1 | 9.0 |
| 227915_at | 1.4 | 0.2 | 1.0 | 0.2 | 0.6 | 0.0 | 18.4 | 3.1 |
| 238600_at | 1.4 | 0.1 | 1.2 | 0.1 | 1.2 | 0.0 | 14.5 | 1.2 |
| 238423_at | 1.4 | 0.1 | 1.2 | 0.1 | 1.3 | 0.1 | 9.8 | 1.9 |
| 236341_at | 1.4 | 0.1 | 1.2 | 0.1 | 1.2 | 0.0 | 14.6 | 3.7 |
| 204015_s_at | 1.4 | 0.2 | 1.0 | 0.3 | 0.7 | 0.0 | 19.7 | 3.5 |
| 234970_at | 1.4 | 0.1 | 1.5 | 0.1 | 1.2 | 0.1 | 10.1 | 4.6 |
| 223229_at | 1.4 | 0.1 | 1.4 | 0.2 | 1.4 | 0.1 | 13.2 | 5.4 |
| 205005_s_at | 1.4 | 0.1 | 1.3 | 0.1 | 1.7 | 0.2 | 6.2 | 1.2 |
| 228722_at | 1.4 | 0.1 | 1.3 | 0.1 | 1.4 | 0.1 | 2.9 | 0.3 |
| 203764_at | 1.3 | 0.1 | 1.2 | 0.1 | 1.2 | 0.0 | 14.5 | 7.6 |
| 234973_at | 1.3 | 0.1 | 1.2 | 0.1 | 1.1 | 0.1 | 5.6 | 0.8 |
| 206980_s_at | 1.3 | 0.1 | 0.9 | 0.1 | 1.0 | 0.1 | 6.9 | 0.5 |
| 214551_s_at | 1.3 | 0.1 | 1.1 | 0.1 | 1.2 | 0.0 | 15.1 | 5.3 |
| 220684_at | 1.3 | 0.1 | 1.0 | 0.2 | 0.8 | 0.0 | 13.3 | 1.8 |
| 204917_s_at | 1.3 | 0.1 | 1.1 | 0.1 | 1.5 | 0.1 | 2.8 | 0.8 |
| 205790_at | 1.3 | 0.1 | 1.0 | 0.2 | 1.0 | 0.1 | 10.3 | 1.4 |
| 218585_s_at | 1.3 | 0.1 | 1.2 | 0.1 | 1.2 | 0.0 | 12.0 | 7.6 |
| 34726_at | 1.3 | 0.1 | 1.0 | 0.0 | 1.5 | 0.3 | 2.4 | 0.3 |
| 204962_s_at | 1.3 | 0.1 | 1.2 | 0.1 | 1.2 | 0.0 | 22.6 | 11.8 |
| 1558102_at | 1.3 | 0.1 | 1.0 | 0.1 | 1.2 | 0.1 | 3.6 | 0.7 |
| 206589_at | 1.3 | 0.1 | 1.2 | 0.1 | 0.8 | 0.0 | 11.2 | 1.2 |
| 230536_at | 1.3 | 0.1 | 1.0 | 0.1 | 0.9 | 0.0 | 112 | 3.2 |
| 226299_at | 1.2 | 0.0 | 1.3 | 0.1 | 0.9 | 0.0 | 2.3 | 0.1 |
| 1558304_s_at | 1.2 | 0.1 | 1.0 | 0.1 | 1.2 | 0.1 | 6.1 | 0.7 |
| 212772_s_at | 1.2 | 0.1 | 1.0 | 0.2 | 1.1 | 0.1 | 2.2 | 0.2 |
| 206804_at | 1.2 | 0.1 | 0.9 | 0.0 | 0.8 | 0.0 | 15.5 | 1.7 |
| 212022_s_at | 1.2 | 0.1 | 1.0 | 0.1 | 1.1 | 0.1 | 15.3 | 7.2 |
| 203418_at | 1.2 | 0.0 | 1.1 | 0.1 | 1.2 | 0.0 | 10.9 | 6.0 |
| 235476_at | 1.2 | 0.1 | 1.2 | 0.1 | 1.2 | 0.0 | 7.9 | 0.5 |
| 1554539_a_at | 1.2 | 0.1 | 0.9 | 0.1 | 1.1 | 0.1 | 8.0 | 0.9 |
| 209840_s_at | 1.2 | 0.1 | 1.1 | 0.0 | 1.1 | 0.0 | 88.5 | 12.3 |
| 227868_at | 1.2 | 0.1 | 1.1 | 0.1 | 1.2 | 0.1 | 6.3 | 0.8 |
| 219315_s_at | 1.2 | 0.1 | 1.0 | 0.1 | 1.1 | 0.1 | 2.3 | 0.1 |
| 229268_at | 1.2 | 0.1 | 1.3 | 0.2 | 1.2 | 0.1 | 4.5 | 0.4 |
| 216693_x_at | 1.2 | 0.1 | 1.0 | 0.2 | 1.3 | 0.1 | 3.8 | 1.0 |
| 209642_at | 1.2 | 0.0 | 1.2 | 0.1 | 1.2 | 0.0 | 9.5 | 5.9 |
| 222039_at | 1.2 | 0.1 | 1.0 | 0.1 | 0.9 | 0.1 | 13.3 | 7.4 |
| 226661_at | 1.2 | 0.0 | 1.2 | 0.1 | 1.2 | 0.0 | 9.4 | 4.1 |
| 238846_at | 1.2 | 0.0 | 1.2 | 0.1 | 1.2 | 0.1 | 7.2 | 3.5 |
| 235816_s_at | 1.2 | 0.0 | 1.2 | 0.1 | 1.2 | 0.0 | 10.7 | 0.9 |
| 227313_at | 1.2 | 0.0 | 1.0 | 0.1 | 1.1 | 0.0 | 2.1 | 0.1 |
| 207183_at | 1.2 | 0.0 | 12 | 0.1 | 1.2 | 0.0 | 5.3 | 1.1 |
| 207315_at | 1.2 | 0.0 | 1.2 | 0.1 | 1.2 | 0.0 | 7.2 | 3.0 |
| 1553423_a_at | 1.2 | 0.0 | 1.2 | 0.1 | 1.2 | 0.0 | 2.7 | 0.5 |
| 206039_at | 1.2 | 0.0 | 1.2 | 0.1 | 1.2 | 0.0 | 20.3 | 2.9 |
| 206999_at | 1.2 | 0.0 | 1.2 | 0.1 | 1.2 | 0.0 | 10.5 | 4.9 |
| 207723_s_at | 1.2 | 0.0 | 1.2 | 0.1 | 1.2 | 0.0 | 13.8 | 6.0 |
| 208511_at | 1.2 | 0.0 | 1.2 | 0.1 | 1.2 | 0.0 | 3.6 | 0.6 |
| 210837_s_at | 1.2 | 0.0 | 1.2 | 0.1 | 1.2 | 0.0 | 2.0 | 0.3 |
| 211207_s_at | 1.2 | 0.0 | 1.2 | 0.1 | 1.2 | 0.0 | 3.1 | 0.5 |
| 211861_x_at | 1.2 | 0.0 | 1.2 | 0.1 | 1.2 | 0.0 | 1.8 | 0.2 |
| 235266_at | 1.2 | 0.0 | 1.2 | 0.1 | 1.2 | 0.0 | 4.4 | 1.1 |
| 241871_at | 1.2 | 0.0 | 1.2 | 0.1 | 1.2 | 0.0 | 12.4 | 3.4 |
| 225387_at | 1.1 | 0.1 | 0.7 | 0.2 | 0.9 | 0.1 | 5.5 | 0.3 |
| 209464_at | 1.1 | 0.0 | 1.1 | 0.1 | 1.1 | 0.0 | 14.6 | 7.5 |
| 204040_at | 1.1 | 0.1 | 1.0 | 0.2 | 1.2 | 0.1 | 6.7 | 0.7 |
| 206506_s_at | 1.1 | 0.0 | 1.0 | 0.2 | 1.3 | 0.1 | 2.7 | 0.2 |
| 215797_at | 1.1 | 0.0 | 1.2 | 0.1 | 1.2 | 0.0 | 4.1 | 1.0 |
| 207460_at | 1.1 | 0.1 | 0.9 | 0.1 | 0.8 | 0.0 | 10.9 | 1.0 |
| 221582_at | 1.1 | 0.1 | 1.1 | 0.0 | 0.9 | 0.0 | 4.3 | 0.4 |
| 227686_at | 1.1 | 0.1 | 1.1 | 0.0 | 1.1 | 0.1 | 3.5 | 0.5 |
| 218726_at | 1.1 | 0.1 | 1.1 | 0.1 | 1.0 | 0.0 | 11.7 | 4.1 |
| 212728_at | 1.1 | 0.0 | 1.1 | 0.0 | 1.2 | 0.1 | 1.7 | 0.1 |
| 236717_at | 1.1 | 0.1 | 1.1 | 0.1 | 0.9 | 0.0 | 7.0 | 0.7 |
| 222777_s_at | 1.1 | 0.1 | 1.1 | 0.1 | 1.2 | 0.1 | 4.3 | 1.0 |
| 206518_s_at | 1.1 | 0.0 | 1.1 | 0.1 | 1.1 | 0.0 | 4.2 | 1.4 |
| 209197_at | 1.1 | 0.1 | 0.8 | 0.1 | 1.1 | 0.1 | 4.5 | 0.8 |
| 212949_at | 1.1 | 0.0 | 0.9 | 0.1 | 0.9 | 0.0 | 10.4 | 3.5 |
| 213623_at | 1.1 | 0.1 | 1.0 | 0.1 | 1.1 | 0.1 | 3.8 | 0.9 |
| 208524_at | 1.1 | 0.0 | 1.1 | 0.1 | 1.1 | 0.0 | 13.7 | 8.0 |
| 218434_s_at | 1.1 | 0.0 | 1.1 | 0.2 | 1.3 | 0.1 | 1.7 | 0.2 |
| 202179_at | 1.0 | 0.0 | 1.0 | 0.0 | 1.1 | 0.0 | 2.4 | 0.2 |
| 209602_s_at | 1.0 | 0.1 | 1.1 | 0.1 | 0.9 | 0.1 | 5.2 | 0.7 |
| 204159_at | 1.0 | 0.1 | 1.1 | 0.0 | 0.8 | 0.1 | 3.7 | 1.5 |
| 226627_at | 1.0 | 0.1 | 0.9 | 0.1 | 0.9 | 0.1 | 3.9 | 0.4 |
| 224832_at | 1.0 | 0.1 | 1.0 | 0.1 | 1.2 | 0.0 | 3.3 | 0.5 |
| 220576_at | 1.0 | 0.0 | 1.1 | 0.1 | 1.0 | 0.0 | 3.3 | 1.3 |
| 220613_s_at | 1.0 | 0.1 | 1.0 | 0.1 | 1.3 | 0.1 | 3.6 | 1.1 |
| 210763_x_at | 1.0 | 0.1 | 0.9 | 0.1 | 0.9 | 0.1 | 10.4 | 1.4 |
| 203565_s_at | 0.9 | 0.0 | 1.2 | 0.1 | 1.2 | 0.1 | 2.4 | 0.3 |
| 225842_at | 0.9 | 0.0 | 0.8 | 0.0 | 1.1 | 0.1 | 3.8 | 1.0 |
| 214950_at | 0.9 | 0.0 | 0.9 | 0.1 | 0.6 | 0.0 | 9.9 | 1.3 |
| 220941_s_at | 0.9 | 0.1 | 0.7 | 0.1 | 0.9 | 0.1 | 6.8 | 1.2 |
| 208406_s_at | 0.9 | 0.1 | 0.8 | 0.0 | 0.9 | 0.0 | 12.0 | 1.6 |
| 215195_at | 0.8 | 0.0 | 0.8 | 0.0 | 0.8 | 0.1 | 1.9 | 0.2 |
| 211685_s_at | 0.8 | 0.1 | 0.9 | 0.1 | 0.7 | 0.1 | 2.6 | 0.5 |
| 205171_at | 0.8 | 0.1 | 0.8 | 0.1 | 0.9 | 0.1 | 3.5 | 0.6 |
| 226433_at | 0.8 | 0.1 | 0.8 | 0.1 | 0.7 | 0.1 | 7.4 | 1.2 |
| 227678_at | 0.8 | 0.1 | 0.8 | 0.1 | 0.8 | 0.1 | 3.9 | 0.4 |
| 210426_x_at | 0.7 | 0.1 | 0.7 | 0.0 | 0.7 | 0.0 | 3.9 | 0.9 |

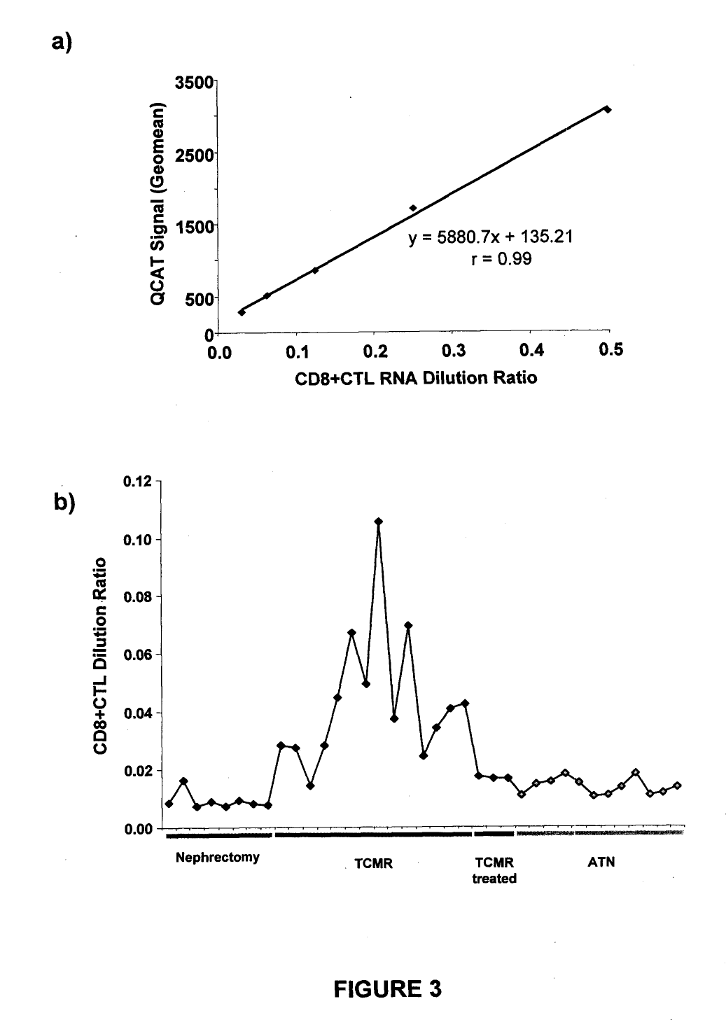

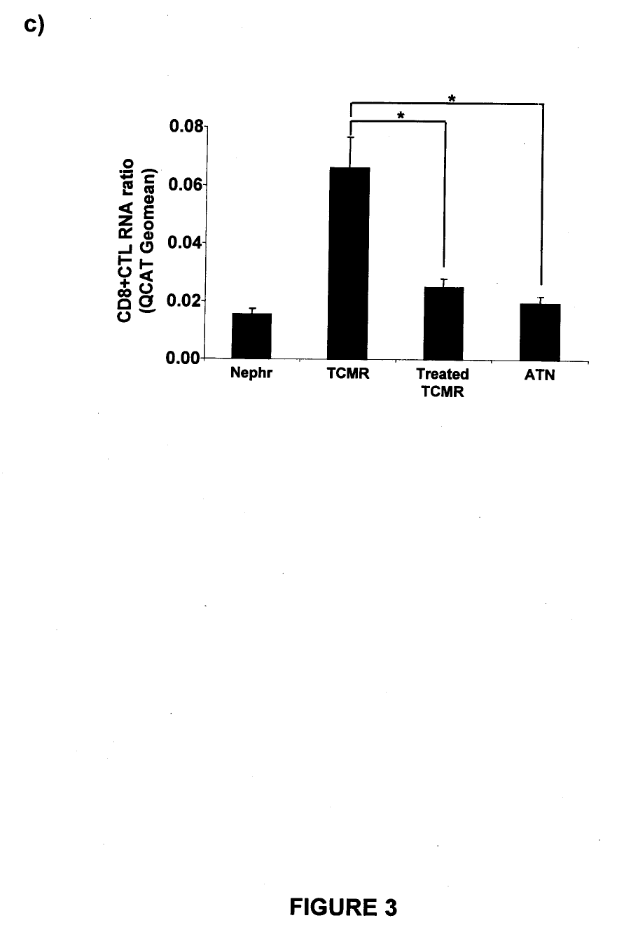

Generation of quantitative CATs: To estimate the CTL burden in kidney tissue, CD8 CATs were used that were highly expressed and that correlated with dilutions of CTL RNA in kidney RNA. These CATs were referred to as quantitative CATs. RNA from a CD8 CTL sample was dissolved into increasing amounts of nephrectomy RNA in five serial two-fold dilutions starting with a 1:1 CTL to nephrectomy ratio. CD8 CATs with a correlation of at least 0.98 between signal intensity and dilution ratio were selected. Transcripts with signal intensities less than 1000 in the 1:1 CTL to nephrectomy dilution ratio were removed. To ensure that the selected transcripts were detectable in TCMR biopsies, only quantitative CATs with a signal value lower than 50 in TCMR cases were removed. The remaining 25 transcripts composed the quantitative CAT list and included cytolytic molecules (GNLY, GZMA, PRF1, GZMK, and GZMB), signaling molecules (CD3D, CD8A, LCK, ITK, STAT4), the NK receptor KLRK1, as well as the effector cytokine IFNG (Table 1). A standard curve of the geomean signal for quantitative CATs versus the CTL RNA dilution ratios was plotted (FIG. 3A). The high correlation (r=0.99) reflects the selection criteria for the transcripts in this set.

Quantitative CAT expression also was examined in CD8 EM cells, which also home to inflamed sites (Masopust et al., J Immunol., 172(8):4875-82 (2004)). Published microarray results (Willinger et al., J Immunol., 175(9):5895-903 (2005)) were available for 15 of 25 quantitative CATs (Table 1). EM T cells expressed similar levels of all of these quantitative CATs as CD8 CTL with values of 2849 for CD8 CTL and 2061 for EM CD8 T cells. These results indicate that quantitative CATs do not distinguish between CTL and EM cells.

Estimation of CTL burden in index cases of TCMR: For quantitative CATs to estimate the CTL burden, they must differentiate between samples predicted to have different CTL content. The quantitative CAT geomean values were compared for TCMR, treated TCMR (which should have a decreased CTL burden), ATN (which are expected to have very low CTL burden), and CD8 CTL, all normalized to nephrectomies. Geomean values differentiated TCMR from treated TCMR and ATN cases (p<0.0005).

The CTL RNA ratio was calculated for individual clinical samples comparing nephrectomy, TCMR, treated TCMR, and ATN (FIG. 3B). A CTL RNA ratio was calculated as the quantitative CAT signal geomean of a sample divided by the geomean for CD8 CTL. The predicted CTL RNA ratios identified high variability within the TCMR group, similar to the highly expressed CATs above. A lower overall CTL burden was evident in both treated TCMR and ATN cases compared to TCMR cases (FIG. 3C). Variability was also observed in the ATN group with some cases having values overlapping TCMR cases.

Unsupervised cluster analysis of all samples in the study and CD8 CTL was performed. Samples clustered according to their similarity to CD8 CTL in the general order: nephrectomy, ATN, treated TCMR, and TCMR from left to right. All TCMR cases, except for one, grouped together and showed the highest predicted CTL burden. Only one case diagnosed as TCMR had a low predicted CTL burden. In support of a decreased CTL burden, histology of this case showed low interstitial infiltrate and tubulitis scores and was diagnosed as having TCMR only on the basis of vasculitis lesions. This represents an uncommon variant of rejection which may identify a potential shortcoming in an empirically defined classification system (Racusen et al., Am J Transplant., 4:1562-1566 (2004)). Cases diagnosed as ATN had low but variable predicted CTL burden and were generally separated into two groups according to a high or low predicted CTL burden. One nephrectomy sample and treated TCMR cases had intermediate predicted CTL burdens and clustered among the ATN cases.

TABLE 2
Expression of quantitative CD8 CATs in nephrectomy, TCMR, treated TCMR, and ATN
cases

| Affymetrix | Gene | | GI | | StdErr | |
|---|---|---|---|---|---|---|
| Probeset | Symbol | Genbank | Number | Nephr | Norm | Signal |
| 205488_at | GZMA | NM_006144 | 6996012 | 0.9 | 0.2 | 24.9 |
| 210164_at | GZMB | J03189 | 338010 | 1.0 | 0.1 | 19.5 |
| 206666_at | GZMK | NM_002104 | 73747815 | 0.9 | 0.1 | 53.3 |
| 205831_at | CD2 | NM_001767 | 31542293 | 0.9 | 0.1 | 60.1 |
| 205758_at | CD8A | AW006735 | 5855513 | 1.0 | 0.1 | 62.9 |
| 204891_s_at | LCK | NM_005356 | 112789545 | 0.9 | 0.2 | 36.1 |
| 213915_at | NKG7 | NM_005601 | 49574503 | 1.0 | 0.1 | 30.0 |
| 205821_at | KLRK1 | NM_007360 | 6679051 | 1.0 | 0.1 | 64.3 |
| 213539_at | CD3D | NM_000732 | 98985799 | 1.0 | 0.1 | 98.0 |
| 207651_at | H963 | NM_013308 | 31377771 | 0.9 | 0.1 | 21.2 |
| 202503_s_at | KIAA0101 | NM_014736 | 71773764 | 0.9 | 0.2 | 36.8 |
| 211339_s_at | ITK | D13720 | 399657 | 1.0 | 0.1 | 16.5 |
| 209671_x_at | TRA@ | M12423 | 338738 | 0.9 | 0.2 | 103.8 |
| 205291_at | IL2RB | NM_000878 | 23238195 | 0.9 | 0.1 | 77.5 |
| 37145_at | GNLY | M85276 | 189229 | 1.0 | 0.1 | 20.5 |
| 214617_at | PRF1 | AI445650 | 4289772 | 1.0 | 0.1 | 84.2 |
| 210140_at | CST7 | AF031824 | 3252857 | 1.0 | 0.1 | 50.4 |
| 216920_s_at | TRGV9 | M27331 | 540458 | 0.9 | 0.1 | 43.6 |
| 210354_at | IFNG | M29383 | 186514 | 1.0 | 0.1 | 17.7 |
| 209773_s_at | RRM2 | BC001886 | 12804874 | 0.9 | 0.2 | 34.1 |
| 211597_s_at | HOP | AB059408 | 13560280 | 0.8 | 0.2 | 100.8 |
| 206118_at | STAT4 | NM_003151 | 21618332 | 1.0 | 0.1 | 35.1 |
| 206974_at | CXCR6 | NM_006564 | 5730105 | 1.0 | 0.1 | 30.3 |
| 203413_at | NELL2 | NM_006159 | 5453765 | 1.0 | 0.1 | 56.2 |
| 218039_at | NUSAP1 | NM_016359 | 59710089 | 0.9 | 0.2 | 51.5 |

| Affymetrix | StdErr | | StdErr | Treated | StdErr | | StdErr |
|---|---|---|---|---|---|---|---|
| Probeset | Raw | TCMR | Norm | TCMR | Norm | ATN | Norm |
| 205488_at | 5.8 | 7.8 | 1.8 | 3.6 | 1.3 | 1.5 | 0.2 |
| 210164_at | 1.4 | 7.3 | 1.8 | 2.0 | 0.7 | 1.3 | 0.1 |
| 206666_at | 7.4 | 7.2 | 1.3 | 2.8 | 0.9 | 1.4 | 0.2 |
| 205831_at | 10.0 | 6.5 | 1.2 | 2.2 | 0.2 | 1.7 | 0.2 |
| 205758_at | 8.3 | 5.9 | 1.1 | 2.5 | 0.2 | 1.2 | 0.1 |
| 204891_s_at | 8.1 | 5.8 | 1.2 | 1.8 | 0.1 | 1.5 | 0.2 |
| 213915_at | 1.9 | 4.9 | 0.9 | 2.4 | 0.4 | 1.7 | 0.2 |
| 205821_at | 7.8 | 4.9 | 0.6 | 2.6 | 0.7 | 1.7 | 0.1 |
| 213539_at | 7.2 | 4.9 | 0.9 | 1.7 | 0.0 | 1.3 | 0.1 |
| 207651_at | 3.6 | 4.7 | 1.1 | 1.8 | 0.0 | 1.1 | 0.1 |
| 202503_s_at | 7.1 | 4.5 | 0.7 | 2.5 | 0.4 | 2.8 | 0.5 |
| 211339_s_at | 2.2 | 4.4 | 1.0 | 1.3 | 0.2 | 1.2 | 0.1 |
| 209671_x_at | 17.3 | 4.1 | 0.6 | 1.8 | 0.2 | 1.4 | 0.1 |
| 205291_at | 6.4 | 3.7 | 0.6 | 1.8 | 0.1 | 1.4 | 0.1 |
| 37145_at | 1.2 | 3.7 | 0.8 | 2.1 | 0.7 | 1.7 | 0.3 |
| 214617_at | 7.9 | 3.6 | 0.7 | 1.9 | 0.4 | 1.4 | 0.1 |
| 210140_at | 5.1 | 3.6 | 0.4 | 1.9 | 0.2 | 1.1 | 0.1 |
| 216920_s_at | 7.7 | 3.6 | 0.7 | 1.2 | 0.1 | 1.2 | 0.2 |
| 210354_at | 1.6 | 3.3 | 0.8 | 1.5 | 0.1 | 1.1 | 0.0 |
| 209773_s_at | 7.3 | 3.1 | 0.7 | 2.0 | 0.2 | 1.9 | 0.4 |
| 211597_s_at | 29.5 | 2.9 | 0.6 | 1.2 | 0.0 | 2.1 | 0.8 |
| 206118_at | 4.2 | 2.8 | 0.4 | 1.1 | 0.1 | 1.4 | 0.1 |
| 206974_at | 2.1 | 2.1 | 0.3 | 1.5 | 0.1 | 0.8 | 0.0 |
| 203413_at | 7.2 | 2.0 | 0.3 | 0.8 | 0.1 | 0.9 | 0.1 |
| 218039_at | 7.0 | 2.0 | 0.3 | 1.2 | 0.1 | 1.7 | 0.2 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting tissue rejection, said method comprising determining whether or not tissue transplanted into a human contains cells having a human transplant rejection profile, wherein the presence of said cells indicates the presence of rejection.

2. The method of claim 1, wherein said tissue is kidney tissue.

3. The method of claim 1, wherein said tissue is a kidney.

4. The method of claim 1, wherein said method comprises using kidney cells obtained from a biopsy to assess the presence or absence of said human transplant rejection profile.

5. The method of claim 1, wherein said determining step comprises analyzing nucleic acids.

6. The method of claim 1, wherein said determining step comprises analyzing polypeptides.

7. A method for assessing tissue rejection, said method comprising determining the mean expression of quantitative CD8 CATs in cells from tissue transplanted into a human, wherein a greater difference between said mean expression of quantitative CD8 CATs and the mean of corresponding reference levels indicates a greater extent of rejection.

8. The method of claim 7, wherein said tissue is kidney tissue.

9. The method of claim 7, wherein said tissue is a kidney.

10. The method of claim 7, wherein said method comprises using kidney cells obtained from a biopsy to determine said mean expression of quantitative CD8 CATs.

11. The method of claim 7, wherein said determining step comprises analyzing nucleic acids.

12. The method of claim 7, wherein said determining step comprises analyzing polypeptides.
